(19) United States
(12) Patent Application Publication
(10) Pub. No.: US 2020/0236983 A1
(43) Pub. Date: Jul. 30, 2020

(54) COMPOSITIONS AND METHODS FOR INCREASING MITOCHONDRIAL ACTIVITY

(71) Applicant: Corr-Jensen Inc., New York, NY (US)

(72) Inventors: Peter J. Miller, Broomfield, CO (US); Matthew Hesse, New York, NY (US)

(21) Appl. No.: 16/773,333

(22) Filed: Jan. 27, 2020

Related U.S. Application Data
(60) Provisional application No. 62/796,819, filed on Jan. 25, 2019.

(51) Int. Cl. A23L 33/16; A61K 33/06

(52) U.S. Cl. CPC A23L 33/16; A61K 45/06; A61K 33/06

(57) ABSTRACT

Provided are compositions and methods for increasing mitochondrial activity, increasing exercise performance, or combinations thereof. The compositions include one or more performance-enhancing components that increase ATP generation within the mitochondria leading to improved exercise performance, preventing athletic fatigue, such as sustaining energy levels and avoiding a subsequent energy level crash, or any combination thereof, when consumed by a subject.

### CROSS REFERENCE TO RELATED APPLICATIONS

This application depends from and claims priority to U.S. Patent Application No. 62/796,819 filed Jan. 25, 2019, the entire contents of which are incorporated herein by reference.

### FIELD

The invention relates to exercise and sports performance. More specifically, compositions and methods are provided that improve mitochondrial activity and other aspects of cellular health and performance by promoting movement of molecular hydrogen into cells.

### BACKGROUND

Nutritional requirements play a key role improving athlete endurance and performance. With the entrance of Gatorade® and other similar sports drinks beginning in the mid-1960s more attention has been paid to understanding the role of various nutrients on human performance in athletic events. More recent entrants into the category have focused on providing caffeine and other stimulants in order to enhance performance.

Typical sports drinks and energy supplying supplements have historically focused on obtaining quick bursts of energy during exercise or other periods of strenuous exertion. However, these quick bursts of energy are for a short duration, thereby requiring the user to consume additional supplements or rely on self-contained energy stores. Additionally, these quick bursts of energy may cause undue stress on cellular health, which may lead to a decrease in performance if typical supplements are excessively ingested over periods of time. In these cases, immunity may become compromised and overtraining may occur leading to long-term health and performance impairments.

Thus, there is a need in the art to provide formulations that improve performance in order to enhance both short-term and long-term athletic performance, recovery and health.

### SUMMARY

It is understood that both the following summary and the detailed description are exemplary and explanatory and are intended to provide further explanation of the disclosure as claimed. Neither the summary nor the description that follows is intended to define or limit the scope of the disclosure to the particular features mentioned in the summary or description.

Provided are methods and compositions that enhance athletic performance by increasing the mitochondrial activity within the subject. The methods and compositions as provided herein increase the ability of cellular mitochondria to produce adenosine triphosphate (ATP), an essential requirement for athletic performance for both short and long term duration. The methods and compositions as provided herein increase the presence of or transport of molecular hydrogen into cells thereby stimulating mitochondrial activity, optionally along with other aspects of improving or sustaining athletic performance.

A performance-enhancing composition as provided herein optionally includes at least one first performance-enhancing component and optionally an acid. The performance-enhancing composition optionally includes and inner capsule (or bead) and an outer capsule wherein the inner capsule or bead is housed within the outer capsule. If present, the inner capsule or bead and outer capsule are optionally separated by a barrier, which is optionally formed by the exterior of the inner capsule or bead. Optionally, the inner capsule is in the form of a time or immediate release bead that is packaged in an outer capsule, optionally within one or more oils (e.g. edible oils or room temperature fluid triglycerides).

Also provided are methods of increasing mitochondrial activity in a subject, optionally within the skeletal muscle of a subject. The method includes administering a performance-enhancing composition including a formulation including at least one first performance-enhancing component and optionally an acid. The performance-enhancing composition optionally includes and inner capsule (or bead) and an outer capsule wherein the inner capsule is housed within the outer capsule. If present, the inner capsule or bead and outer capsule are optionally separated by a barrier, which is optionally formed by the exterior of the inner capsule or bead. Optionally, the inner capsule is in the form of a time or immediate release bead that is packaged in an outer capsule, optionally within one or more oils (e.g. edible oils or room temperature fluid triglycerides).

In one aspect, the performance-enhancing composition includes an immediate release powder comprising at least one first performance-enhancing component and optionally a trace mineral complex, stimulant, testosterone booster, alkaline agent, or combinations thereof. The immediate release powder is soluble or partially soluble in an aqueous medium.

A performance-enhancing composition is optionally administered to a subject orally, optionally before exercise.

### DETAILED DESCRIPTION

Provided are methods and compositions that increase mitochondrial activity and boost exercise performance in a subject. The methods and compositions are intended to increase the level of molecular hydrogen (H₂) in a cell of a subject that then acts by one or more signaling pathways to increase ATP production. Increases in ATP production lead to improved exercise performance in the subject.

The compositions as provided herein include at least one performance-enhancing component for immediate or sustained introduction of molecular hydrogen to cells thereby improving mitochondrial activity before, during, and/or after periods of physical activity, maintaining vasodilation during and after a workout, stimulating muscle synthesis and repair over a period of time, and/or preventing athletic fatigue, such as sustaining energy levels and avoiding a subsequent energy level crash, or any combination thereof, when consumed by a subject. The compositions as provided herein according to some aspects may include one or more agents that increase alkalinity (e.g. prevent acid buildup) in a subject or portion thereof, optionally by the introduction of an alkaline agent (e.g. beta-alanine, carnosine). According to some aspects, the compositions as provided herein may synergistically improve testosterone levels, thereby enhancing performance and recovery, prime cell activity before periods of strenuous activity, or reduce recovery periods after periods of strenuous activity.

As such, a performance-enhancing composition according to some aspects as provided herein includes a formulation including at least one first performance-enhancing component and optionally a trace mineral and/or macromineral. It is believed that mitochondrial activity is increased as a result of the performance-enhancing composition by introducing or increasing an amount of molecular hydrogen to the cells.

Thus, through the action of the one or more performance-enhancing components alone or in combination with the one or more acids, the instantly disclosed performance-enhancing composition may promote the introduction of molecular hydrogen to cells, thereby improving mitochondrial activity, maintaining vasodilation during and after a workout, stimulating muscle synthesis and repair over an extending period of time, and/or preventing athletic fatigue, such as sustaining energy levels and avoiding a subsequent energy level crash, when consumed by a subject. Without being bound by theory, it is believed that some performance-enhancing components as provided herein promote the introduction of molecular hydrogen to or into cells. This introduction of molecular hydrogen upregulates one or more signaling pathways (such as NPF2 and/or PGC-1α pathways), thereby improving mitochondrial health and the performance of a subject during strenuous activities.

Mitochondrial activity as used herein is optionally measured by the level of adenosine triphosphate (ATP) generation over unit time. Mitochondrial health is necessary for efficient and robust ATP generation within cells. ATP generation is driven by a proton gradient across the mitochondrial inner membrane. The compositions as used herein according to some aspects promote mitochondrial health and increase ATP generation by improving the supply of molecular hydrogen to the cells through the unique combination of components that can optionally increase alkalinity in a subject (or portion thereof, e.g. plasma, cytoplasmic, etc.) as well as generation of molecular hydrogen that is more readily moved across cellular membranes where the effects of molecular hydrogen on cellular and mitochondrial health occur.

ATP generation may be measured using a bioluminescence assay from mitochondrial preparations of skeletal muscle obtained from a subject. Such methods are described in Wibom R and Hultman E. ATP production rate in mitochondria isolated from microsamples of human muscle. Am J Physiol Endocrinol Metab 259: E204-E209, 1990. An increase in ATP generation may be related to the level of ATP generation prior to administration of a performance-enhancing composition as provided herein to the subject, or to a standard baseline level as established from a control population. A baseline level of ATP generation may be 6.6+/−1.3 mmol/min/kg in sedentary subjects to 11.0+/−2.2 mmol/min/kg in highly active subjects as established by Wibrom and Hultman. A composition as provided herein optionally increases mitochondrial ATP generation by 1 mmol/min/kg to 10 mmol/min/kg or greater. Optionally, ATP generation is increased by 1 percent relative to baseline or prior to administration of a performance-enhancing composition as provided herein, optionally 2 percent relative to baseline, optionally 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 200 percent or more relative to baseline or prior to administration of a composition as provided herein.

A performance-enhancing composition is optionally packaged in the form of an inner capsule or bead and an outer capsule, wherein the outer capsule and inner capsule or bead are optionally separated by a barrier. The barrier between the outer capsule and the inner capsule of the formulation is optionally formed by the exterior of the inner capsule. Optionally, the barrier is a surface of a bead or plurality of beads packaged within the outer capsule. Alternatively, or in addition, a performance-enhancing composition is in the form of an immediate release powder whereby upon ingestion or upon contact with water will immediately begin to produce molecular hydrogen or will be immediately available to increase exercise performance in a subject. In other aspects, a performance-enhancing composition is in the form of an immediate release powder with extended release granules that are tailored to deliver one or more performance-enhancing components to a subject over a desired course of time. Any combination of the forgoing is also envisioned.

In some aspects, a performance-enhancing composition includes an immediate release powder including at least one first performance-enhancing component and optionally a trace mineral and/or macromineral. The immediate release powder is optionally soluble or partially soluble in an aqueous medium. It is believed that mitochondrial activity is increased as a result of the performance-enhancing composition by introducing or increasing an amount of molecular hydrogen to the cells.

As used herein, the term “exercise performance” means performance in athletics. Performance means strong, precise, controlled movements over the time desired by an athlete to achieve a particular result of strength, speed, power and/or precision. “Athlete” or “subject” is herein defined as a mammal that performs such movements, either in competition or for recreation. Athletes illustratively include cyclists, swimmers, bodybuilders, racehorses, racing dogs and the like. An increase in athletic performance is measured as higher power output, more stamina, faster speed, or increased caloric output over the course of time, optionally in combination with precision of movement or an increase in frequency of performance or movements.

The term “fatigue” as used herein means the inability to maintain a consistent level of peak athletic performance for a desired period of time. Fatigue is this definition may be due to the exhaustion of energy sources to metabolize, buildup of toxic metabolites in muscle and the like, but not due to lack of sleep, metabolic disease, or illness.

As used herein, the term “performance-enhancing component” is intended to encompass a chemical composition that functions to enhance exercise performance through one or more mechanisms, prevent reduction in exercise performance, prevent fatigue, or combinations thereof.

As such, a performance-enhancing composition as provided herein according to some aspects includes a formulation comprising at least one first performance-enhancing component. In some aspects, a performance-enhancing component is optionally a composition that will cause the generation of molecular hydrogen when contacted with an aqueous material such as water, plasma, cytoplasm, etc. Such performance-enhancing components optionally include one or more minerals, optionally one or more macrominerals. Macrominerals include calcium, phosphorus, magnesium, sodium, potassium, chloride, and sulfur. In some aspects, the macromineral is present in the composition from 0 weight percent (wt. %) to 40 wt. %. Optionally, a macromineral is present in a performance-enhancing composition at from 0.1 wt. % to 20 wt. %, 1 wt. % to 10 wt. %, 1 wt. % to 7 wt. %, 1 wt. % to 5 wt. %.

In some aspects, a performance-enhancing composition includes a magnesium-based composition. A magnesium-based composition is a compound that includes magnesium (e.g. elemental magnesium) or a collection of compounds of which magnesium is a component at a level that is suitable for increasing molecular hydrogen in a subject. Optionally, a magnesium-based composition includes magnesium at a weight percent relative to other compounds that is equal to or greater than the weight percent of each of the other compounds. A magnesium-based composition is optionally magnesium in one or more forms. Illustrative examples of a magnesium-based composition include but are not limited to magnesium bisglycinate chelate, dimagnesium phosphate, magnesium bisglycinate chealate, trimagnesium citrate, magnesium oxide, magnesium chloride, magnesium stearate, or any combination thereof. In some aspects, a magnesium-based composition includes magnesium obtained from a natural source, optionally sea salt, algae (e.g. Lithothamnion calcaneum), seaweed, or other natural source.

In some aspects, the magnesium-based composition is present in the performance-enhancing composition from 0 weight percent (wt. %) to 20 wt. % of the performance-enhancing composition, or any range value therebetween. For example the magnesium-based composition is present in the performance-enhancing composition at a weight percent of 1 wt. % to 18 wt. %, 2 wt. % to 16 wt. %, 13 wt. % to 15 wt. %, 1 wt. % to 14 wt. %, 1 wt. % to 13 wt. %, 1 wt. % to 12 wt. %, 1 wt. % to 11 wt. %, 1 wt. % to 10 wt. %, or any value or range therebetween. In aspects, the one or more magnesium-based compositions of the formulation are optionally located in an inner capsule or bead, the outer capsule, or both. Optionally, the one or more magnesium-based compositions are in the form of an immediate release powder, or packaged into an immediate release or sustained release bead.

In some aspects, a performance-enhancing composition includes one or more acids. It is believed that the inclusion of one or more acids acts synergistically to enhance the ability of a magnesium-based composition to generate molecular hydrogen in an aqueous environment or to further enhance the ability to increase the rate or amount of molecular hydrogen entering a cell of the body. An acid is illustratively one or more of citric acid, malic acid, tartaric acid, or combinations thereof. In some aspects, an acid may provide additional magnesium relative to a magnesium-based compound. As such, an acid is optionally trimagnesium citrate. The amount of the one or more acids is optionally from 0.01 wt. % to 70 wt. % or any value or range therebetween. Optionally, the amount of the one or more acids is from 0.01 wt. % to 20 wt. %, optionally 0.1 wt. % to 10 wt. %.

In some aspects, a performance-enhancing composition includes one or more trace minerals, or a collection of trace minerals, optionally alone or in combination with one or more macrominerals. There are over 72 trace minerals known such as those that may be found in seawater of the North Atlantic Ocean. Illustrative trace minerals include, but are not limited to boron, iron, manganese, copper, iodine, zinc, cobalt, fluoride, and selenium. Optionally, trace minerals may be obtained from a natural source, optionally sea salt, algae, seaweed, or other such natural sources. An illustrative example of a composition that is both rich in magnesium as well as trace minerals includes REDMUNDS Ancient Sea Salt, or AQUAMIN (magnesium focused trace mineral complex from red algae).

In certain aspects, the performance-enhancing composition comprises from 0 wt. % to 60 wt. % of trace mineral, or any range value therebetween. For example the trace mineral is present in the composition at a weight percent of 0.01 wt. % to 55 wt. %, 0.1 wt. % to 55 wt. %, 1 wt. % to 55 wt. %, 10 wt. % to 50 wt. %, 15 wt. % to 45 wt. %, 17 wt. % to 40 wt. %, 20 wt. % to 37 wt. %, 22 wt. % to 36 wt. %, 24 wt. % to 34 wt. %, 26 wt. % to 30 wt. %, or any value or range therebetween.

A performance-enhancing composition as provided herein optionally further includes one or more alkaline promoting compounds (e.g. alkaline agent) that function to increase or substantially maintain the alkalinity within a subject or portion thereof. Increasing alkalinity acts to buffer lactic acid produced as a result of exercise thereby increasing the ability for additional sustained exertion without undergoing the “burn” due to lactic acid build up within cells or other compartments within a mammalian body. Any alkaline agent with a pKa greater than 5.0 may optionally be used. Illustrative examples of an alkaline agent include but are not limited to members of the bicarbonate family, phosphate family, acetate family, or ammonia family, amino acids, carnitine, among others. One illustrative example of an alkaline agent is sodium bicarbonate. Another illustrative example of an alkaline agent is creatine. Another illustrative example of an alkaline agent is carnitine. Another illustrative example of an alkaline agent is beta-alanine. The amount of alkaline agent within a performance-enhancing composition is optionally from 0.01 wt % to 5 wt % or any value or range therebetween. An alkaline promoting compound is optionally located in the inner capsule or bead, the outer capsule, in an immediate release powder, in a time release bead, or any combination thereof.

A performance-enhancing composition optionally includes one or more vitamins, or a collection of vitamins. The one or more vitamins may be water-soluble, fat-soluble, or a combination thereof. Illustrative vitamins include, but are not limited to, vitamin A, biotin, vitamin B3 (optionally in the form of nicinaminde/nicotinamide available from DSM), vitamin B6, vitamin B12 (optionally in the form of methylcobalamin available from Anmar), vitamin C, vitamin D3 (optionally in the form of cholecalciferol), vitamin E, and folic acid. The one or more vitamins of a formulation are optionally located in the inner capsule or bead, the outer capsule, in an immediate release powder, in a time release bead, or any combination thereof; optionally with certain vitamins in the outer capsule and certain vitamins in the inner capsule or bead. The amount of one or more vitamins is optionally from 0.01 wt % to 15 wt % or any value or range therebetween.

A composition optionally includes one or more amino acids, or a collection of amino acids. The one or more amino acids may include essential amino acids, non-essential amino acids, branched amino acids, and the like. Illustrative amino acids include, but are not limited to, beta-alanine or derivative thereof (optionally CARNOSYN beta-alanine available from Natural Alternatives Intl.), arginine (optionally in the form of arginine silicate inositol available as Nitrosigine from Nutrition 21, or agmatine sulfate available from Parchem) glutamine (available from Kyowa Hakko), theanine (optionally in the form of L-theanine), creatine (optionally in the form of creatine HCL available from Pharmline), carnitine (optionally in the form of L-carnitine; optionally in the form of acetyl-L-carnitine HCl), glycine, trimethyl glycine, tyrosine, leucine (available from Glanbia or Danisco), isoleucine (available from Glanbia), valine (available from Glanbia), citrulline (optionally in the form of citrulline malate available from Creative Compounds; optionally in the form of L-citrulline DL-malate at a 1:1 or 2:1 ratio), N-acetyl L-tyrosine (available from Cepham), norvaline (optionally in the form of L-novaline, available from Cepham), and branched chain amino acids (optionally in the form of Pepform 2:1:1 BCAA containing a 2:1:1 ratio of leucine, isoleucine and valine, available from Glanbia), among others or derivatives thereof. An amino acid is optionally located in the inner capsule or bead, the outer capsule, in an immediate release powder, in a time release bead, or any combination thereof.

A performance-enhancing composition optionally includes one or more proteins comprised of one or more amino acids or amino acid chains. The one or more amino acids and/or proteins are optionally located in the inner capsule or bead, the outer capsule, or both; optionally with certain amino acids and/or proteins in the outer capsule and certain amino acids and/or proteins in the inner capsule or bead. The amount of one or more amino acids and/or proteins is optionally from 0.01 wt % to 60 wt % or any value or range therebetween. As an example, the amount of theacrine in the inner capsule or bead is optionally from 0.01 wt % to 20 wt % or any value or range therebetween.

Optionally, a composition may include an amino acid that is additionally a performance-enhancing component. Illustrative examples of amino acids or amino acid derivatives that are also performance-enhancing components include, but are not limited to, creatine, magnesium bisglycinate, and citrulline malate. In certain aspects, creatine is optionally located in the inner capsule or bead, the outer capsule, or both. The amount of creatine is optionally 0.01 wt % to 20 wt % or any value or range therebetween. In certain aspects, citrulline malate is optionally located in the inner capsule, the outer capsule, or both. The amount of citrulline malate is optionally from 0.01 wt % to 40 wt % or any value or range therebetween.

Optionally, a composition may include an amino acid that is additionally both a performance-enhancing component and a source of magnesium. An illustrative example of an amino acid that is also a performance-enhancing component and a source for magnesium is magnesium bisglycinate, for example in chelated form. In certain aspects, magnesium bisglycinate is optionally located in the inner capsule or bead. The amount of magnesium bisglycinate in the inner capsule or bead is optionally from 0.01 wt % to 5 wt % or any value or range therebetween.

A composition optionally includes 1-alpha glycerylphosphorylcholine. In certain aspects, 1-alpha glycerylphosphorylcholine is optionally located in the inner capsule or bead, the outer capsule, in an immediate release powder, in a time release bead, or any combination thereof. The amount of 1-alpha glycerylphosphorylcholine in the composition is optionally 0.01 wt % to 5 wt % or any value or range therebetween.

A composition optionally includes one or more fatty acids. Optionally, the one or more fatty acids include one or more essential fatty acids. In certain embodiments, one or more fatty acids is optionally located in the inner capsule or bead, the outer capsule, in an immediate release powder, in a time release bead, or any combination thereof. The amount of the one or more fatty acids is optionally 0.01 wt % to 5 wt % or any value or range therebetween.

A performance-enhancing component optionally includes one or more stimulants, or a collection of stimulants. Illustrative examples of stimulants include caffeine (optionally 1,3,7-trimethylxanthine available from Mitsubishi; optionally in the form of purple caffeine 85% CR beadlets), ephedrine, theacrine (such as from Camellia sinesis; optionally available as Teacrine from Double Wood Supplements), creatine, or forskholin. In certain embodiments, the one or more stimulants is optionally located in the inner capsule or bead, the outer capsule, in an immediate release powder, in a time release bead, or any combination thereof. The amount of the one or more stimulants is optionally 0.01 wt % to 25 wt % or any value or range therebetween. In certain embodiments, caffeine is optionally located in the outer capsule. The amount of caffeine is optionally 0.01 wt % to 25 wt % or any value or range therebetween. The amount of caffeine in the outer capsule is optionally 0.01 wt % to 40 wt % or any value or range therebetween. Theacrine is optionally located in the inner capsule or bead. The amount of theacrine is optionally 0.01 wt % to 5 wt % or any value or range therebetween. The amount of theacrine in the inner capsule or bead is optionally 0.01 wt % to 20 wt % or any value or range therebetween.

A performance-enhancing component optionally includes one or more testosterone boosters, or a collection of testosterone boosters. Illustrative examples of testosterone boosters include shilajit, Eurycoma longifolia extract, fenugreek extract, boron citrate, dehydroepiandrosterone (DHEA), Tribulus terrstris extract, and ashwaganda (Withania somnifera) extract. In certain embodiments, the one or more testosterone boosters are optionally located in the inner capsule or bead, the outer capsule, in an immediate release powder, in a time release bead, or any combination thereof. The amount of the one or more testosterone boosters is optionally 0.01 wt % to 30 wt % or any value or range therebetween. In certain embodiments, ashwaganda is optionally located in the outer capsule. The amount of ashwaganda is optionally 0.01 wt % to 10 wt % or any value or range therebetween. The amount of ashwaganda in the outer capsule is optionally 0.01 wt % to 25 wt % or any value or range therebetween. Shilajit is optionally located in the inner capsule or bead. The amount of shilajit is optionally 0.01 wt % to 10 wt % or any value or range therebetween. The amount of shilajit in the inner capsule or bead is optionally 0.01 wt % to 35 wt % or any value or range therebetween. Eurycoma longfolia extract is optionally located in the inner capsule or bead. The amount of eurycoma longifolia extract is optionally 0.01 wt % to 10 wt % or any value or range therebetween. The amount of eurycoma longifolia extract in the inner capsule or bead is optionally 0.01 wt % to 35 wt % or any value or range therebetween.

A performance-enhancing component optionally includes boron. Boron may be in any desired form, optionally as a boron citrate. Boron citrate is optionally located in the inner capsule or bead. The amount of boron citrate is optionally 0.01 wt % to 3 wt % or any value or range therebetween. The amount of boron citrate in the inner capsule or bead is optionally 0.01 wt % to 5 wt % or any value or range therebetween.

A performance-enhancing component optionally includes one or more components of metabolic pathways. Illustrative examples of components of metabolic pathways include pyruvate, pyruvic acid, citric acid cyclic intermediates, and betaine (optionally in the form of betaine anhydrous available from Danisco), or derivatives thereof, among others, or combinations thereof. In certain embodiments, the one or more components of metabolic pathways are optionally located in the inner capsule or bead, the outer capsule, in an immediate release powder, in a time release bead, or any combination thereof. The amount of the one or more components of metabolic pathways is optionally 0.01 wt % to 25 wt % or any value or range therebetween.

A performance-enhancing component optionally includes one or more plant components, such as an essential oil or plant extract. Illustrative examples of plant components include citrus aurantium, grape seed extract, theacrine extract (such as from Camellia sinesis; optionally available as Teacrine from Double Wood Supplements), black pepper Piper nigrum extract (optionally as 95% piperine black pepper extract; optionally available from Indena and/or as Black Pepper Fruit Extract from BioPerine), ashwagandha extract (optionally KSM66 from Ixoreal), mushroom extract (such as from Lion's mane mushroom of Hericium erincaeus (optionally in powder form) or portabello mushroom of Agaricus bisporus), green tea leaf extract (optionally available as Vaso6 from Serious Nutrition Solutions; optionally including theacrine, for example available as Teacine from Double Wood Supplements), yohimbe bark extract from Pausinystalia johimbe (optionally in powder form at 8% before mixed with the other components), yohimbine from Pausinystalia johimbe or Aspidosperma quebracho-blanco, extract from Eurycoma longifolia (optionally available as LJ100 from Tongkat Ali), extract from Huperzia firmmoss (such as Huperzine A extract), extract from Berberis plants (such as berberine extract, optionally in the form of berberine hydrochloride), extract from Coleus forskohlii (such as forskolin extract), extract from shilajit (optionally in the form of Primavie), Mucuna pruriens extract (such as L-DOPA), extracts from cruciferous vegetables (exemplary cruciferous vegetables include broccoli, cabbage, brussels sprouts, cauliflower, bok choy, and kale; optionally, the cruciferous vegetable extract includes one or more glucosinolates, diindolymethane, and/or indole-3-carbinol), and hot pepper extract (optionally including capsaicin and/or capsaicinoids; optionally in the form of Capsimax) or the like, or a derivative of any of the foregoing. In certain embodiments, the one or more plant components is optionally located in the inner capsule or bead, the outer capsule, in an immediate release powder, in a time release bead, or any combination thereof. The amount of the one or more plant components is optionally 0.01 wt % to 25 wt % or any value or range therebetween.

A performance-enhancing composition optionally includes Astragalus membranaceus or an extract therefrom. In certain aspects, Astragalus membranaceus is optionally located in the inner capsule or bead, the outer capsule, in an immediate release powder, in a time release bead, or any combination thereof. The amount of Astragalus membranaceus in the composition is optionally 0.01 wt % to 5 wt % or any value or range therebetween.

A performance-enhancing composition optionally includes Panax notoginseng (optionally in the form of Astragin available from N Liv Science). In certain embodiments, Panax notoginseng is optionally located in the inner capsule or bead, the outer capsule, in an immediate release powder, in a time release bead, or any combination thereof. The amount of Panax notoginseng in the composition is optionally 0.01 wt % to 5 wt % or any value or range therebetween.

In certain aspects, the performance-enhancing composition, optionally the outer capsule of the performance-enhancing composition, can include one or more edible oils or components thereof, such as fatty acids and medium chain triglycerides. An edible oil is optionally packaged within an outer capsule and houses within it a suspension of inner capsules or beads that include one or more performance-enhancing components. In some aspects, an edible oil is a fish oil, or optionally a bioactive component thereof. As used herein, “fish oils” are oils that are obtained either directly or indirectly from one or more aquatic life forms. Fish oil can be derived from fresh or salt water fish or shellfish. In certain aspects, fish oils are obtained from oily fish. Fish oils are high in one or more of omega-3 fatty acids, such as docosahexaneoic acid, eicosapentaenoic acid docosapentaenoic acid, eicosatetraenoic acid, moroctic acid and heneicosapentenoic acid relative to non-fish oils. Omega-3 fatty acids are beneficial for prevention of cardiovascular pathology, for reversal of atherosclerosis, for inhibition of tumor formation, and for regulation of cholesterol. In certain aspects, the one or more fish oils of the outer capsule of the formulation comprises docosahexaneoic acid and eicosapentaenoic acid. The amount of fish oil in the formulation is optionally 0.01 wt % to 99 wt % or any value or range therebetween. The amount of fish oil in the outer capsule is optionally 0.01 wt % to 60 wt % or any value or range therebetween. The amount of medium chain triglycerides in the performance-enhancing composition is optionally 0.01 wt % to 50 wt % or any value or range therebetween. The amount of medium chain triglycerides in the outer capsule is optionally 0.01 wt % to 90 wt % or any value or range therebetween.

It is appreciated that some aspects may include more than one edible oil, optionally 2, 3, 4, 5, 6, or more edible oils or bioactive components thereof. Illustrative additional or substitutable edible oils include, but are not limited to vegetable oils, such as, evening primrose oil, black currant seed oil, borage oil, borage seed oil, safflower oil, safflower seed oil, sunflower oil, sunflower seed oil, sesame seed oil, peanut oil, walnut oil, almond oil, olive oil, olive seed oil, avocado oil, avocado seed oil, pumpkin seed oil, corn oil, cod liver oil, soy oil, soybean oil, coconut oil, palm oil, palm kernel oil, rapeseed oil, flaxseed (linseed) oil, cotton seed oil, tung oil, palmolein oil, mustard seed oil, oiticica oil and castor oil, arachidonic acid, leichitin, and conjugated linoleic acids combinations thereof. Edible oils are commercially available from sources known by those of skill in the art. In certain aspects, the one or more edible oils can be in a liquid or paste-like state at 25° C.

In certain aspects, the one or more edible oils of the outer capsule of the formulation can range from about 0.5 wt % to about 90 wt %, including any value or range therebetween. In other aspects, the one or more edible oils of the outer capsule of the formulation can range from about 5 wt % to about 50 wt %, including any value or range therebetween.

In some aspects, the performance-enhancing composition can be a formulation comprising an inner capsule or bead and an outer capsule, wherein the inner capsule or bead and the outer capsule are separated by a barrier. The barrier may be the surface of the inner capsule or bead and serves to prevent solubilizing the inner capsule or bead components within a liquid (e.g. edible oil, triglyceride, etc.) housed within the outer capsule. In certain aspects, the performance-enhancing composition of the instant disclosure can be a formulation comprising an inner capsule or bead and an outer capsule, whereby the inner capsule or bead is housed within the outer capsule, and further comprise a barrier between the inner capsule and the outer capsule that is formed by the exterior of the inner capsule or bead.

In some aspects, the at least one performance-enhancing component is suitable to improve mitochondrial performance via promoting the introduction of an amount of molecular hydrogen within the subject or a portion thereof, such as enhancing exercise performance during and after a workout and stimulating muscle synthesis and repair over an extending period of time, and/or preventing athletic fatigue, such as sustaining energy levels and avoiding a subsequent energy level crash. The at least one performance-enhancing component of the formulation is or includes illustratively one or more of: a macromineral, a magnesium-based compound (such as trimagnesium citrate, magnesium carbonate, magnesium bisglycinate chelate, magnesium citrate, dimagnesium phosphate, a liposomal magnesium oxide complex (such as Sucrosomial® Magnesium, commercially available from MayPro), a magnesium mineral complex (such as Aquamin MG from Aquamin)), a trace mineral, a silicon-based compound (such as calcium silicate or silicon dioxide), at least one acid (such as citric acid, malic acid, or tartaric acid), plant extract, 1-alpha glycerylphosphorylcholine, a testosterone booster, a stimulant, a component of a metabolic pathway, or any combination thereof or derivatives of any of the foregoing.

In certain aspects as provided herein, the performance-enhancing composition may further include an inner capsule or bead and an outer capsule. The inner capsule or bead and outer capsule optionally house one or more active agents such as a performance-enhancing component and or a mineral. The barrier separating the inner capsule or bead and outer capsule is optionally provided to allow for the formulation to contain components with distinct properties (illustratively, one of the inner capsule or bead or the outer capsule contains predominantly hydrophobic components, and the other of the inner capsule or bead or the outer capsule alternatively contains predominantly hydrophilic components; illustratively, one of the inner capsule or bead or the outer capsule contains predominantly water-soluble components, and the other of the inner capsule or the outer capsule alternatively contains predominantly oil-soluble components). The barrier between the inner capsule or bead and the outer capsule is optionally formed by the exterior of the inner capsule or bead.

In some aspects, the outer capsule of the formulation optionally houses within the outer capsule of or more of a: edible oil (illustratively a fish oil or a medium chain triglyceride), vitamin (illustratively, a fat-soluble vitamin such as vitamin D), plant component, flavoring agent, preservative, testosterone booster, and stimulant. In some aspects, the outer capsule of the formulation comprises one or more inner capsules or beads (beadlets) suspended in an oil phase, wherein the oil phase comprises one or more of: an edible oil, a vitamin, a plant component, a flavoring agent, a preservative, a stimulant or combinations thereof. In some aspects, the beadlets of the outer capsule comprise a stimulant (illustratively caffeine or ashwagandha) and one or more performance-enhancing compositions. In some aspects, the outer capsule includes an exterior that separates the components of the outer capsule from the environment.

In some aspects, the inner capsule or bead of the formulation is separated from the outer capsule of the formulation by a barrier, wherein the outer capsule houses one or more components that surrounds the barrier and the barrier surrounds the inner capsule or bead. In some aspects, the inner capsule or bead is completely encapsulated within the outer capsule, with the barrier separating the inner capsule and the outer capsule. In some aspects, the barrier separates the inner capsule or bead and the outer capsule such that components of the inner capsule cannot move into the other components housed within the outer capsule, and components housed within the outer capsule cannot move into the inner capsule. In some aspects, the barrier between the inner capsule or bead and the outer capsule is formed by the exterior of the inner capsule or bead.

In some aspects, the inner capsule or bead of the performance-enhancing composition optionally comprises of or more of a: macromineral (illustratively magnesium or a magnesium-based complex), trace mineral (illustratively boron), vitamin (illustratively, a water-soluble vitamin such as vitamin B12), stimulant (illustratively theacrine), testosterone booster (illustrative examples include boron citrate and eurycoma longifolia), plant component, flavoring agent, preservative, alkaline agent, 1-alpha phosphorylcholine, silicon-based compound, components of a metabolic pathway, excipient, stimulant (illustratively forskholin), or any combination thereof. In aspects, the inner capsule or bead of the formulation predominantly comprises water-soluble components.

In some aspects, a performance-enhancing composition comprises an exterior of an inner capsule that is formed by a gelatin capsule. In aspects, the components of the inner capsules are located within the gelatin capsule. In aspects, the gelatin capsule is configured to contain 50-500 mg of components, including any value or range therebetween. In aspects, the gelatin capsule is configured to contain 50-100 mg of components, including any value or range therebetween. In some aspects, the gelatin capsule is configured to contain 100-200 mg of components, including any value or range therebetween. In aspects, the gelatin capsule is configured to contain 200-300 mg of components, including any value or range therebetween. In aspects, the gelatin capsule is configured to contain 300-400 mg of components, including any value or range therebetween. In aspects, the gelatin capsule is configured to contain 400-500 mg of components, including any value or range therebetween.

In certain aspects, the performance-enhancing composition includes a formulation comprising at least one performance-enhancing component in an inner capsule or bead, an outer capsule, or both, such that the formulation provides sustained mitochondrial activity improvement, an extended performance burst, or combinations thereof.

In some aspects, the performance-enhancing composition can be an immediate release powder comprising at least one first performance-enhancing component. In certain aspects, the performance-enhancing supplement of the instant disclosure can be a component of an immediate release powder that further includes extended release granules.

In some aspects, the at least one performance-enhancing component within the immediate release powder is suitable to improve mitochondrial performance via promoting the introduction of an amount of molecular hydrogen, such as enhancing exercise performance during and after a workout and stimulating muscle synthesis and repair over an extending period of time, and/or preventing athletic fatigue, such as sustaining energy levels and avoiding a subsequent energy level crash. The at least one performance-enhancing component of the immediate release powder is illustratively one or more of: a macromineral, a magnesium-based compound (such as trimagnesium citrate, magnesium carbonate, magnesium bisglycinate chelate, magnesium citrate, dimagnesium phosphate, a liposomal magnesium oxide complex (such as Sucrosomial® Magnesium, commercially available from MayPro), a magnesium mineral complex (such as Aquamin MG from Aquamin), a trace mineral, a silicon-based compound (such as calcium silicate or silicon dioxide), at least one acid (such as citric acid, malic acid, or tartaric acid), plant extract, 1-alpha glycerylphosphorylcholine, a testosterone booster, a stimulant, a component of a metabolic pathway, or combinations thereof or derivatives of any of the foregoing. In certain aspects, the at least one performance-enhancing component of the immediate release powder is an uncoated performance-enhancing component.

In certain aspects as provided herein, the performance-enhancing composition may further include extended release granules that allow for extended release of one or more active agents such as a performance-enhancing component and or a trace mineral. The extended release granules may include a core. The core may include at least one second performance-enhancing component and one or more fatty materials. The extended release granules may be formulated for sustained release, such that the extended release granules provide a sustained performance.

In certain aspects, the extended release granules include at least one performance-enhancing component formulated for sustained release, delayed release, or both, such that the extended release granules provides sustained mitochondrial activity improvement, an extended performance burst, or combinations thereof. The extended release granules may comprise a core, with the core of the extended release granules comprising the at least one second performance-enhancing component and one or more fatty materials.

In certain aspects, the performance-enhancing composition includes an immediate release powder comprising at least one performance-enhancing component and extended release granules comprising a core, optionally including a second performance-enhancing component. The core of the extended release granules comprises at least one performance-enhancing component and one or more fatty materials to provide a desired buoyancy in an aqueous medium. In certain aspects, the performance-enhancing composition or portion thereof is soluble in an aqueous medium. In some aspects, the performance-enhancing composition is reconstitutable into a dispersion medium, which is optionally an aqueous solution.

The performance-enhancing composition optionally includes an immediate release powder comprising at least one performance-enhancing component. The term “immediate release” is the release of the at least one performance-enhancing component from the immediate release powder of the powder blend where the rate of release is not retarded by means of a controlled release matrix, coating, or other such means, and where the components of the at least one performance-enhancing component from the immediate release powder are designed such that, upon ingestion, maximum exposure of the at least one performance-enhancing component from the immediate release powder to body tissues occurs in the minimum period of time. As described herein, an “immediate release” powder preferably releases at least one performance-enhancing component in less than or equal to 10 minutes, in less than or equal to 5 minutes, in less than or equal to 3 minutes, in less than or equal to 2 minutes, or in less than or equal to 1 minute. The at least one performance-enhancing component of the immediate release powder is optionally water soluble or partially water soluble so as to be effectively and immediately suspendable in a dispersion medium (e.g. an aqueous medium) upon dilution at a temperature at which the dispersion medium is in liquid form, at a concentration of the performance-enhancing component as to provide an in vivo concentration effective to function to improve athletic performance, and for a time sufficient to allow consumption. A time sufficient to allow consumption may be less than or equal to 1 minute, less than or equal to 2 minutes, less than or equal to 5 minutes, less than or equal to 10 minutes, less than or equal to 15 minutes, less than or equal to 20 minutes, less than or equal to 30 minutes, less than or equal to 45 minutes, less than or equal to 1 hour, less than or equal to 2 hours, or any other reasonable time for consumption of the dispersion medium containing the performance-enhancing component.

The magnesium-based composition may allow for greater biological availability of phospholipid micelles made from sucrose esters of fatty acid, thereby promoting the introduction of an amount of molecular hydrogen to cells. Such sucrose esters of fatty acid help increase the absorption and bioavailability of minerals (such as magnesium), which play a role in a large number of cellular reactions. Specifically, the cellular reactions may include the biosynthesis of lipids, proteins, ATP, or nucleic acids that—optionally together—improve mitochondrial activity.

As such, in aspects, the magnesium-based composition optionally includes at least one fatty acid ester. The fatty acid ester may include, esterified fatty acid methyl esters of sucrose. In certain embodiments, the magnesium-based composition comprises tricalcium phosphate. In certain embodiments, the magnesium-based compound comprises dimagnesium phosphate. In certain embodiments, the magnesium-based compound comprises magnesium bisglycinate chelate. In certain embodiments, the magnesium-based compound comprises trimagnesium citrate anhydrous.

In some embodiments, the at least one first performance-enhancing component comprises from 0 wt. % to 20 wt. % of the magnesium-based composition, or any range value therebetween. For example the magnesium-based composition is present in the at least one first performance-enhancing component at a weight percent of 1 wt. % to 18 wt. %, 2 wt. % to 16 wt. %, 3 wt. % to 15 wt. %, 4 wt. % to 14 wt. %, 5 wt. % to 13 wt. %, 6 wt. % to 12 wt. %, 7 wt. % to 11 wt. %, 8 wt. % to 10 wt. %, or any value or range therebetween.

In certain embodiments, the composition comprises from 0 wt. % to 60 wt. % of one or more trace minerals, or any range value therebetween. For example the trace mineral is present in the performance-enhancing composition at a weight percent of 5 wt. % to 55 wt. %, 10 wt. % to 50 wt. %, 15 wt. % to 45 wt. %, 17 wt. % to 40 wt. %, 20 wt. % to 37 wt. %, 22 wt. % to 36 wt. %, 24 wt. % to 34 wt. %, 26 wt. % to 30 wt. %, or any value or range therebetween.

The trace mineral may include any suitable mineral. Suitable minerals may include, but are not limited to boron, bromide, carbonate, calcium, silicon, nitrogen, selenium, phosphorus, iodide, chromium, manganese, titanium, rubidium, cobalt, copper, antimony, molybdenum, strontium, zinc, nickel, tungsten, germanium, scandium, vanadium, tellurium, tin, lanthanum, yttrium, silver, gallium, bismuth, zirconium, cerium, cesium, gold, beryllium, hafnium, samarium, terbium, europium, gadolinium, dysprosium, thorium, holmium, lutetium, erbium, ytterbium, neodymium, praseodymium, niobium, tantalum, thallium, rhenium, indium, palladium, or combinations thereof.

In some embodiments, the at composition comprises from 0 wt % to 20 wt. % calcium silicate, or any range value therebetween. For example calcium silicate is present in the composition at a weight percent of 2 wt. % to 18 wt. %, 3 wt. % to 16 wt. %, 4 wt. % to 14 wt. %, 5 wt. % to 12 wt. %, 6 wt. % to 10 wt. %, 7 wt. % to 9 wt. %, or any value or range therebetween.

In further embodiments, the composition comprises from 0 wt. % to 20 wt. % silicon dioxide, or any range value therebetween. For example silicon dioxide is present in the at least one first performance-enhancing component at a weight percent of 2 wt. % to 18 wt. %, 3 wt. % to 16 wt. %, 4 wt. % to 14 wt. %, 5 wt. % to 12 wt. %, 6 wt. % to 10 wt. %, 7 wt. % to 9 wt. %, or any value or range therebetween.

In additional embodiments, the composition comprises from 0 wt. % to 60 wt. % citric acid, or any range value therebetween. For example citric acid is present in the at least one first performance-enhancing component at a weight percent of 5 wt. % to 55 wt. %, 10 wt. % to 50 wt. %, 15 wt. % to 45 wt. %, 17 wt. % to 40 wt. %, 20 wt. % to 37 wt. %, 22 wt. % to 36 wt. %, 24 wt. % to 34 wt. %, 26 wt. % to 30 wt. %, or any value or range therebetween.

In other embodiments, the composition comprises from 0 wt. % to 25 wt. % malic acid, or any range value therebetween. For example malic acid is present in the at least one first performance-enhancing component at a weight percent of 2 wt. % to 23 wt. %, 4 wt. % to 21 wt. %, 6 wt. % to 19 wt. %, 8 wt. % to 18 wt. %, 10 wt. % to 15 wt. %, 12 wt. % to 13 wt. %, or any value or range therebetween. In some embodiments, the malic acid is in the form of DL-malic acid.

In some embodiments, the composition comprises from 0 wt. % to 10 wt. % tartaric acid, or any range value therebetween. For example tartaric acid is present in the at least one first performance-enhancing component at a weight percent of 1 wt. % to 9 wt. %, 2 wt. % to 7 wt. %, 3 wt. % to 6 wt. %, 3 wt. % to 5 wt. %, 3 wt. % to 4 wt. %, or any value or range therebetween.

The performance-enhancing composition may include an alkaline agent. Without being bound by theory, it is believed that the alkaline agent prevents acid buildup in a cell after the at least one performance-enhancing component is ingested by a user. As such, mitochondrial performance is improved before and/or after periods of strenuous activity. Moreover, recovery period is shortened. Suitable alkaline agents include any beta amino acids. In some embodiments, the alkaline agent comprises beta-alanine, beta-leucine, or combinations thereof. In certain embodiments, the alkaline agent is beta-alanine.

Moreover, the performance-enhancing composition may enhance testosterone levels by increasing the production of testosterone by enhancing Leydig cell activity in the testis. Without being bound by theory, increased testosterone levels may improve performances before and/or after periods of strenuous activity, and also shorten the recovery period. As such, the performance-enhancing composition improves Leydig cell activity and recovery period, and general mitochondrial health. Illustrative examples of testosterone boosters include shilajit, Eurycoma longifolia extract, fenugreek extract, boron citrate, dehydroepiandrosterone (DHEA), Tribulus terrstris extract, and ashwaganda (Withania somnifera) extract.

The performance-enhancing composition may be delivered to a user in any suitable form. Suitable forms of delivery include, but are not limited to, capsules, pills, powders, liquids, functional foods optionally in the form of a bar, chewable tablets, gummies, or combinations thereof. In certain embodiments, the performance-enhancing composition is administered by a powder. In certain embodiments, the performance-enhancing composition is administered by a formulation including an inner capsule and an outer capsule, wherein the outer capsule and the inner capsule are separated by a barrier. The barrier between the outer capsule and the inner capsule of the formulation is optionally formed by the exterior of the inner capsule.

The at least one performance-enhancing component is present to provide an in vivo concentration effective to function to improve athletic performance, such as improving mitochondrial activity during and after a workout and stimulating muscle synthesis and repair over an extending period of time, and/or preventing athletic fatigue, such as sustaining energy levels, avoiding a subsequent energy level crash, and increasing testosterone levels. In certain aspects, the at least one performance-enhancing component of the immediate release powder is optionally present at a weight percent of the performance-enhancing immediate and extended release powder blend of 5 wt. % to 50 wt. %, or any value or range therebetween. For example, the at least one performance-enhancing component is optionally present at a weight percent of 5 wt. % to 10 wt. %, 10 wt. % to 15 wt. %, 15 wt. % to 20 wt. %, 20 wt. % to 25 wt. %, 25 wt. % to 30 wt. %, 30 wt. % to 35 wt. %, 35 wt. % to 40 wt. %, 40 wt. % to 45 wt. %, 45 wt. % to 50 wt. %, or any value or range therebetween.

In some aspects, the mineral included in the composition is a trace mineral or a macromineral. In aspects, the mineral included in the composition is selected from the group consisting of boron, calcium, chromium, cobalt, copper, fluorine, iodine, iron, magnesium, manganese, molybdenum, nickel, selenium, silicon, sodium, vanadium, zinc, or combinations thereof.

In certain aspects, the composition optionally includes one or more excipients including but not limited to, e.g., one or more of a sweetener, a preservative, sodium citrate; silica; flavorants, colorants, preservatives, or other components. The choice of which such materials to use, if any, and the amounts to be utilized are considered to be within the abilities of one of skilled in the art, in view of the disclosure herein.

Exemplary sweeteners may include those sweeteners well known in the art, including both natural and artificial sweeteners. Thus, exemplary sweeteners may include water-soluble sweetening agents such as monosaccharides, disaccharides, and polysaccharides such as xylose, ribose, glucose, mannose, galactose, fructose, high fructose corn syrup, dextrose, sucrose, sugar, maltose, partially hydrolyzed starch, or corn syrup solids and sugar alcohols such as sorbitol, xylitol, mannitol and mixtures thereof. Additional exemplary sweeteners include optionally sugar or sugar substitute (e.g. sucralose (1,6-Dichloro-1,6-dideoxy-β-D-fructofuranosyl-4-chloro-4-deoxy-α-D-galactopyranoside), aspartame, acesulfame potassium, and the like).

Exemplary preservatives may include sodium benzoate, sodium bicarbonate, benzoic acid, potassium sorbate, sea salt (optionally in the form of ancient sea salt available as Redmond's Real Sea Salt), salts of edetate (also known as salts of ethylenediaminetetraacetic acid, or EDTA, such as disodium EDTA), carnosic acid, parabens (e.g., methyl, ethyl, propyl or butyl-hydroxybenzoates, etc.), and sorbic acid. Other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3-PDTA); 2,2-ethylenedioxybis[ethyliminodi(acetic acid)] (EGTA); 1,10-bis(2-pyridylmethyl)-1,4,7,10-tetraazadecane (BPTETA); ethylenediamine (EDAMINE); Trans-1,2-diaminocyclohexane-N,N,N′,N′-tetraacetic acid (CDTA); ethylenediamine-N,N′-diacetate (EDDA); phenazine methosulphate (PMS); 2,6-Dichloro-indophenol (DCPIP); Bis(carboxymethyl)diaza-18-crown-6 (CROWN); porphine; chlorophyll; dimercaprol (2,3-Dimercapto-1-propanol); citric acid; tartaric acid; fumaric acid; malic acid (optionally in the form of DL-malic acid); and salts thereof can be utilized as preservatives. Each preservative must be evaluated in each formulation to assure the compatibility and efficacy of the preservative. Methods for evaluating the efficacy of preservatives in compositions and formulations are known to those skilled in the art.

Exemplary flavorings may include both natural and artificial flavors, and mints such as peppermint, menthol, artificial vanilla, vanilla extract, ginger extract (optionally in the form of ginger CO2 Extract), chocolate, cinnamon, citrus (optionally including D-limonene oil), various fruit flavors, both individual and mixed, tea flavors, sweetness modifiers, essential oils (i.e. thymol, eucalyptol, menthol, theacrine, and methyl salicylate), pie flavors (e.g., pumpkin pie, lemon pie, apple pie), herbs (e.g., rosemary, lemongrass, lavender, jasmine, lemon verbena; optionally including carnosic acid) and extracts thereof, and the like. Exemplary fruit flavorings may include both natural and artificial flavors, such as blueberry, raspberry, strawberry, grape, lemon, lime, cherry, mango, grapefruit, coconut, apple, pineapple, and the like. Flavoring agents may be utilized to provide an appealing flavor to the compositions, mask other flavors of the compositions (e.g., a vegan protein masker), enhance the sweetness and/or savoriness of the compositions, and the like.

Exemplary colorants may include both natural and artificial colorants such as spirulina extract, turmeric power, fruit powder, vegetable powder, cocoa powder, paprika extract, beetroot, and the like. Exemplary colors that may be provided by one or more colorants include red, blue, yellow, green, orange, purple, pink, gray, black, white, brown, variations thereof, and the like.

In some aspects, the at least one first performance-enhancing component of the performance-enhancing composition may include a viscosity modifier. In certain aspect, the viscosity modifier may include one or more of guar gum, xanthan gum, and silica.

The performance-enhancing composition of the instant disclosure may further include an inner capsule or bead that is in the form of immediate release granules, extended release granules, delayed release granules, or a combination of immediate release, extended and delayed release granules. In some aspects, the extended release granules, delayed release granules, or both extended and delayed release granules are formulated to be neutrally buoyant or become buoyant when exposed to a dispersion medium, such as an aqueous medium. In certain aspects, the extended release granules, delayed release granules, or both extended and delayed release granules are formulated to be neutrally buoyant or become buoyant when exposed to an aqueous solution. Thus, when the extended release granules, delayed release granules, or both extended and delayed release granules are exposed to a dispersion medium, such as an aqueous medium, the granules exhibit a neutral buoyancy and are suspendable, without either sinking or floating. In certain aspects, the extended release granules, delayed release granules, or both extended and delayed release granules will remain suspended once dispersed in an aqueous medium, without either sinking or floating within the first 15 seconds of agitation.

In some aspects, the extended release granules, delayed release granules, or both extended and delayed release granules have a bulk density that provides the desired the desired buoyancy of the granules. The term “bulk density,” as used herein, refers to a property of particles and is defined as the mass of many particles of the material divided by the total volume they occupy. A bulk density of from 0.3 g/cc to 0.7 g/cc, including any value or range therebetween, in combination with other factors such a particle size, morphology, surface tension, pH, etc, provides the desired neutral buoyancy of the extended release granules, delayed release granules, or both extended and delayed release granules in an aqueous medium. Thus, the extended release granules, delayed release granules, or both extended and delayed release granules may have a bulk density of 0.3 g/cc to 0.35 g/cc, 0.35 g/cc to 0.40 g.cc, 0.40 g/cc to 0.45 g/cc, 0.45 g/cc to 0.50 g/cc, 0.50 g/cc to 0.55 g/cc, 0.55 g/cc to 0.60 g/cc, 0.60 g/cc to 0.65 g/cc, 0.65 g/cc to 0.70 g/cc, and 0.70 g/cc to 0.75 g/cc in an aqueous medium, including any value or range therebetween. The bulk density of the extended release granules, delayed release granules, or both extended and delayed release granules will vary, and depends of the entirety of the formulation (e.g. the components of the powder blend or the powder blend dispersed in a dispersion medium). For example, when the powder blend is dissolved in a dispersion medium, such variation can be due to the dissolved solids from the immediate release powder of the powder blend. As such, the higher the amount of dissolved solids in the dispersion medium (e.g. aqueous medium), the actual density of the dispersion medium can increase. Further, the viscosity modifiers that can be included in the immediate release powder, once dispersed in a dispersion medium, can act to suspend the extended release granules, delayed release granules, or both extended and delayed release granules. Additionally, other factors that may vary within the immediate release powder, which ultimately affects the dispersion medium once dissolved, can affect the final buoyancy of the granules. Such factors include pH, hydrophilicity/hydrophobicity, and temperature. All of these factors should be accounted for to ensure that the extended release granules, delayed release granules, or both extended and delayed release granules have a bulk density that provides the desired neutral buoyancy of the granules when dispersed in a dispersion medium, e.g. an aqueous medium.

In certain aspects, the performance-enhancing composition may include extended release granules comprising a core. The core of the extended release granules comprises at least one performance-enhancing component and one or more fatty materials. The term “extended release” refers to the gradual release of the at least one performance-enhancing component from the extended release granules of the powder blend over an extended period of time. With extended release, the rate of release of the at least one performance-enhancing component from the extended release granules is reduced in order to maintain therapeutic activity of the at least one performance-enhancing component for a longer period of time. As described herein, an “extended release” granule preferably releases not less than 80% of the at least one performance-enhancing component in less than or equal to 5 hours, e.g., in less than or equal to 4 hours, in less than or equal to 3 hours, in less than or equal to 2 hours, in less than or equal to 1 hour, in less than or equal to 50 minutes, in less than or equal to 40 minutes, or any value or range therebetween. In certain aspects, an “extended release” granule preferably releases not more than 20% of the at least one performance-enhancing component in 1 hour, in 50 minutes, in 40 minutes, in 30 minutes, in 20 minutes, or any value or range therebetween. In other aspects, an “extended release” granule preferably releases not more than 10% of the at least one performance-enhancing component in 1 hour, in 50 minutes, in 40 minutes, in 30 minutes, in 20 minutes, or any value or range therebetween.

In certain aspects, the performance-enhancing composition can include delayed release granules comprising a core. The core of the delayed release granules comprises at least one performance-enhancing component and one or more fatty materials. The term “delayed release” refers to modified release in which the release of the at least one performance-enhancing component from the extended release granules of the powder blend is delayed after oral administration for a finite period of time after which release of the drug is unhindered.

The extended release granules, delayed release granules, or both extended and delayed release granules can be manufactured using methods of granulating that are known in the art. Such methods include, but are not limited to, dry and wet granulation technology, including fluid bed granulation, high shear granulation, extrusion and spheronization, and spay drying. In certain aspects, the extended release granules, delayed release granules, or both extended and delayed release granules are manufactured using high shear granulation optionally substantially as described in U.S. Pat. Nos. 5,462,747 or 6,953,593.

In some aspects, the core of the extended release granules, delayed release granules, or both extended and delayed release granules can include one or more fatty materials. In some aspects, the one or more fatty materials include low-density fatty materials. In some aspects, the one or more fatty materials include powdered fats, and in certain aspects, low density powdered fats. The one or more powered fats can include hydrogenated vegetable oil (optionally in the form of Serotex K, NF or Serotex HM, NF available from Abitec), stearic acid, fractioned oils, such as fractionated vegetable oils including coconut, palm, etc, medium chain triglycerides, monoglycerides, such as glycerol nehenate, glycerol monosterate, and glycerol esters of long chain fatty acids. Without being bound by theory, the addition of the one or more fatty materials to the core of the extended release granules, delayed release granules, or both extended and delayed release granules can be used to adjust the density of the granule to provide the previously-described neutral buoyancy of the granules in a dispersion medium, such as an aqueous medium.

When one or more fatty materials are present in the core of the extended release granules, delayed release granules, or both extended and delayed release granules, such fatty material preferably comprises up to 50 wt. % of the total formulation, including 0.1 wt. % to 50 wt. %, including 0.1 wt. %, 0.5 wt. %, 1 wt. %, 5 wt. %, 10 wt. %, 20 wt. %, 30 wt. %, 40 wt. %, 50 wt. %, and ranges encompassing and bordered by such amounts. Optionally, the fatty material is present at 15 wt. % to 50 wt. %. The amount of fatty material utilized may be governed, at least in part, by the amounts and physical characteristics of the at performance-enhancing components of an immediate release component and extended or delayed release granules and any optional swellable polymer(s), barrier coatings, and additives, with the object being to achieve a granule formulation which provides the previously-described neutral buoyancy in a dispersion medium, such as an aqueous medium.

The at least one performance-enhancing component of the extended release granules, delayed release granules, or both extended and delayed release granules is present to provide an in vivo concentration effective to function to improve athletic performance, such as maintaining vasodilation during and after a workout, stimulating muscle synthesis and repair over an extending period of time, and/or preventing athletic fatigue, such as sustaining energy levels and avoiding a subsequent energy level crash. In certain aspects, the at least one performance-enhancing component of the extended release granules, delayed release granules, or both extended and delayed release granules is optionally present at a weight percent of the performance-enhancing immediate and extended release powder blend of 5 wt. % to 95 wt. %, or any value or range therebetween. For example, the at least one performance-enhancing component of the extended release granules, delayed release granules, or both extended and delayed release granules is optionally present at a weight percent of 1 wt. % to 15 wt. %, 1 wt. % to 25 wt. %, 10 wt. % to 35 wt. %, 25 wt. % to 45 wt. %, 25 wt. % to 55 wt. %, or any value or range therebetween. Optionally, the at least one performance-enhancing component is present at 1 wt. % to 55 wt. % or any value or range therebetween. Optionally, the at least one performance-enhancing component is present at 0.1 wt. % to 20 wt. %.

In some aspects, the extended release granules, delayed release granules, or both extended and delayed release granules includes the same, different, additional, or fewer at least one performance-enhancing component of relative to the at least one performance-enhancing component of the immediate release powder. The at least one performance-enhancing component of the extended release granules, delayed release granules, or both extended and delayed release granules is suitable to enhance exercise performance, such as maintaining vasodilation during and after a workout and stimulating muscle synthesis and repair over an extending period of time, and/or preventing athletic fatigue, such as sustaining energy levels and avoiding a subsequent energy level crash. The at least one performance-enhancing component of the extended release granules, delayed release granules, or both extended and delayed release granules is illustratively one or more of a: vitamin; beta-alanine or derivative thereof, mineral; protein; amino acid; branched-chain amino acids; Astragalus membranaceus; Panax notoginseng; 1-alpha glycerylphosphorylcholine, carbohydrate (optionally in the form of Astragin available from N Liv Science); fatty acid (optionally essential fatty acid); stimulant (illustratively caffeine, ephedrine, creatine, theacrine or forskholin); pyruvate; citric acid cycle intermediate; betaine (optionally in the form of betaine anhydrous available from Danisco), norvaline (optionally in the form of L-novaline, available from Cepham), one or more plant components such as an essential oil or plant extract (illustratively citrus aurantium, grape seed, theacrine extract (such as from Camellia sinesis; optionally available as Teacrine from Double Wood Supplements), Piper nigrum extract (available from Indena), or ashwagandha extract (optionally KSM66 from Ixoreal)), or a derivative of any of the foregoing.

The ratio of at least one performance-enhancing component of the immediate release powder to the at least one performance-enhancing component of the extended release granules, delayed release granules, or both extended and delayed release granules may be adjusted as desired by one of skill in the formulation art. In some aspects, the ratio of at least one performance-enhancing component of the immediate release powder to the at least one performance-enhancing component of the extended release granules, delayed release granules, or both extended and delayed release granules ranges from 1:1 to 5:1, or any value or range therebetween.

In some aspects, the extended release granules, delayed release granules, or both extended and delayed release granules can comprise a barrier coating. A barrier coat comprises a water-permeable, water-insoluble, non-ionic polymer or co-polymer that confers either extended release or delayed release properties to the granules. In one aspect, the barrier coat can be applied, e.g., as an aqueous suspension, over the extended release granules, delayed release granules, or both extended and delayed release granules, and forms a separate layer thereon. In some aspects, the barrier coat is directly over the extended release granules, delayed release granules, or both extended and delayed release granules and the barrier coat layer, i.e., there are no intervening layers between the barrier coat and the granules. Depending upon the polymeric material selected, the barrier coat polymer or co-polymer may be cured (e.g., poly-vinyl acetate or ethylcellulose-based coatings). In certain aspects, a poly-vinyl acetate based coating may further include a plasticizer. In certain aspects, the barrier coating can comprise poly-vinyl acetate-based coatings, ethylcellulose-based coatings (e.g. SURELEASE™), hydrophobic shellac coatings, or enteric coatings, as are known in the art. Enteric coatings can be used to manufacture delayed release granules. Other barrier coatings can be utilized, e.g., the barrier coatings described in U.S. Pat. Nos. 6,066,334 and 6,046,277, 6,046,277, 6,001,392, US2007/0215511, US2005/232986, US2005/232987 US2005/232993, US2005/266032, and US2003/009971, which are incorporated herein by reference.

The total amount of the barrier coating present may vary within a wide range, preferably from 0.1 wt. % to 20 wt. %, including 1 wt. % to 15 wt. %, 5 wt. % to 15 wt. %, 2 wt. % to 10 wt. %, and 2 wt. % to 7.5 wt. % of the total composition, including 1 wt. %, 2 wt. %, 5 wt. %, 7.5 wt. %, 10 wt. %, 15 wt. %, and 20 wt. % and ranges encompassing and bordered by such amounts. The amount of the barrier coating component(s) present may depend, at least in part, upon the amount and identity of each of the other components present (e.g. the amounts and physical characteristics of the at performance-enhancing components of the immediate release component and extended or delayed release granules and any optional swellable polymer(s), barrier coatings, and additives), and the identity and properties of the particular barrier coating component(s), with the object being to achieve a granule formulation which exhibits extended release or delayed release and which provides the previously-described neutral buoyancy in a dispersion medium, such as an aqueous medium.

In some aspects, the extended release granules, delayed release granules, or both extended and delayed release granules may include one or more swellable polymer that acts to modify, prolong, and/or slow the release over time of the at least one performance-enhancing component from the granules. A “swellable polymer” is a polymer that will swell in the presence of a dispersion medium, such as a fluid. Thus, swellable polymers are capable of absorbing water and physically swelling as a result, with the extent to which a polymer can swell being determined by the molecular weight or degree of crosslinking (for crosslinked polymers). The one or more swellable polymer is capable of swelling dimensionally unrestrained in upon contact with a dispersion medium, such as an aqueous medium. Suitable water-swellable polymers include those polymers that swell in a dimensionally unrestrained manner upon contact with water. Such polymers may also gradually erode over time. Examples of such polymers include polyalkylene oxides, such as polyethylene glycols, particularly high molecular weight polyethylene glycols; cellulose polymers and their derivatives including, but not limited to, methylcellulose, ethylcellulose (e.g. SURELEASE™, available from Colorcon as an aqueous ethyl cellulose dispersion containing water (70.6 wt. %), ethylcellulose (18.8 wt. %), ammonium hydroxide (4.4 wt. %), a medium chain triglyceride (4.0 wt. %), and oleic acid (2.2 wt. %)), hydroxyalkyl celluloses, hydroxymethyl cellulose, hydroxyethyl cellulose, hydroxypropyl cellulose, hydroxypropyl methylcellulose (available from Dow Chemical Company), carboxymethylcellulose, microcrystalline cellulose (available from FMC); polysaccharides and their derivatives; chitosan; poly(vinyl alcohol); xanthan gum; maleic anhydride copolymers; poly(vinyl pyrrolidone); starch and starch-based polymers; maltodextrins; poly(2-ethyl-2-oxazoline); poly(ethyleneimine); polyurethane; hydrogels; crosslinked polyacrylic acids; poly(ethylene oxide); and combinations or blends of any of the foregoing. In certain aspects, the one or more swellable polymers may increase to a size sufficient to be retained in the stomach for an extended period of time.

When the optional one or more swellable polymers is present in the extended release granules, delayed release granules, or both extended and delayed release granules, the total amount present may vary within a wide range, preferably from 0.1 wt. % to 50 wt. %, including 2 wt. % to 40 wt. %, 10 wt. % to 40 wt. %, and 2 wt. % to 20 wt. % of the total composition, including 5 wt. %, 10 wt. %, 15 wt. %, 20 wt. %, 30 wt. %, 40 wt. %, 50 wt. %, and ranges encompassing and bordered by such amounts. The amount of the one or more swellable polymer component present may depend, at least in part, upon the amount and identity of each of the other components present (the amounts and physical characteristics of the at performance-enhancing components of the immediate release component and extended or delayed release granules and any fatty material(s), barrier coatings, and additives), and the identity and properties of the particular polymer(s), with the object being to achieve a granule formulation which exhibits extended release or delayed release and which provides the previously-described neutral buoyancy in a dispersion medium, such as an aqueous medium.

Thus, in certain aspects a performance-enhancing composition is provided that comprises an immediate release powder alone or in combination with extended release granules, the granules comprising a core and a barrier coating layer over the core. The immediate release powder comprises at least one first performance-enhancing component. Optionally, the performance-enhancing component is selected from the group consisting beta-alanine, betaine anhydrous, citrulline malate (optionally in the form of citrulline malate available from Creative Compounds; optionally in the form of L-citrulline DL-malate at a 1:1 or 2:1 ratio; the composition may also optionally contain a citrulline blocker to prevent potential adverse side effects of abundant citrulline), carnitine or derivative thereof, citric acid, malic acid, trace mineral(s), a magnesium-based composition, a plant component or extract (e.g. lions mane mushroom powder, green tea extract, yohimbe bark extract, or black pepper extract), creatine, theanine (optionally in the form of L-theanine), N-acetyl L-tyrosine, caffeine, niacinamide, and bioperine black pepper extract.

The core of the extended release granules optionally comprises at least one second performance-enhancing component, one or more fatty materials, and one or more swellable polymers. The at least one second performance-enhancing component of the core is selected from the group consisting of betaine, caffeine, niacin, N-acetyl L-tyrosine citrus aurantium, alpha yohimbine, a magnesium-based compound, a plant component or extract, or vitamin B12. The one or more fatty material of the core comprises hydrogenated vegetable oil. The one or more swellable polymers of the core are selected from the group consisting of microcrystalline cellulose and hydroxypropyl methylcellulose. The barrier coating layer comprises hydroxypropylmethyl cellulose and ethylcellulose. In certain aspects, the immediate release powder is soluble in an aqueous medium. In some aspects, the bulk density of the extended release granules when dispersed in an aqueous medium, e.g. water, is from 0.3 g/cc to 0.75 g/cc. In certain aspects, the extended release granules will remain suspended once dispersed in an aqueous medium, without either sinking or floating within the first 15 seconds of agitation. Such a composition can be used in processes for enhancing athletic performance or preventing fatigue that include administering the instantly-disclosed composition to a mammalian subject, optionally a human, wherein the administration is performed at a time suitable for enhancing athletic performance or preventing fatigue. In certain aspects, the composition can be administered in the form of an oral suspension.

In another aspect, a performance-enhancing composition is provided that comprises an immediate release powder and extended release granules, the granules comprising a core and a barrier coating layer over the core. The immediate release powder comprises at least one first performance-enhancing component selected from the group consisting leucine, isoleucine, valine, betaine anhydrous, citrulline (optionally in the form of citrulline malate available from Creative Compounds; optionally in the form of L-citrulline DL-malate at a 1:1 or 2:1 ratio; the component may also optionally contain a citrulline blocker to prevent potential adverse side effects of abundant citrulline), carnitine or derivative thereof, citric acid, malic acid, caffeine, a plant component or extract, glutamine, theanine (optionally in the form of L-theanine) and branched chain amino acids, coconut water powder, Astragalus membranaceus and Panax notoginseng, and Piper nigrum fruit extract. The core of the extended release granules comprises at least one second performance-enhancing component that may be the same or different from the first performance-enhancing component, one or more fatty material, and one or more swellable polymers. The at least one second performance-enhancing component of the core is selected from the group consisting of leucine and branched chain amino acids. The one or more fatty material of the core comprises hydrogenated vegetable oil. The one or more swellable polymers of the core are selected from the group consisting of microcrystalline cellulose and hydroxypropyl methylcellulose. The barrier coating layer comprises ethylcellulose. In certain aspects, the immediate release powder is soluble in an aqueous medium. In some aspects, the bulk density of the extended release granules when dispersed in an aqueous medium, e.g. water, is from 0.3 g/cc to 0.75 g/cc. In certain aspects, the extended release granules will remain suspended once dispersed in an aqueous medium, without either sinking or floating within the first 15 seconds of agitation. Such a composition can be used in processes for enhancing athletic performance or preventing fatigue that include administering the instantly-disclosed composition to a mammalian subject, optionally a human, wherein the administration is performed at a time suitable for enhancing athletic performance or preventing fatigue. In certain aspects, the composition can be administered in the form of an oral suspension.

In a further aspect, a performance-enhancing composition is provided that comprises an immediate release powder and extended release granules, the granules comprising a core and a barrier coating layer over the core. The immediate release powder comprises at least one first performance-enhancing component selected from the group consisting of citrulline (optionally in the form of citrulline malate available from Creative Compounds; optionally in the form of L-citrulline DL-malate at a 1:1 or 2:1 ratio; the component may also optionally contain a citrulline blocker to prevent potential adverse side effects of abundant citrulline), agmatine sulfate, arginine silicate inositol, norvaline, niacin, aswagandha extract, and Piper nigrum fruit extract. The core of the extended release granules comprises at least one second performance-enhancing component, one or more fatty material, and one or more swellable polymers. The at least one second performance-enhancing component of the core is selected from the group consisting of norvaline, niacin, and grape seed extract polyphenols. The one or more fatty material of the core comprises hydrogenated vegetable oil. The one or more swellable polymers of the core are selected from the group consisting of microcrystalline cellulose and hydroxypropyl methylcellulose. The barrier coating layer comprises ethylcellulose. In certain aspects, the immediate release powder is soluble in an aqueous medium. In some aspects, the bulk density of the extended release granules when dispersed in an aqueous medium, e.g. water, is from 0.3 g/cc to 0.75 g/cc. In certain aspects, the extended release granules will remain suspended once dispersed in an aqueous medium, without either sinking or floating within the first 15 seconds of agitation. Such a composition can be used in processes for enhancing athletic performance or preventing fatigue that include administering the instantly-disclosed composition to a mammalian subject, optionally a human, wherein the administration is performed at a time suitable for enhancing athletic performance or preventing fatigue. In certain aspects, the composition can be administered in the form of an oral suspension.

In some aspects, the extended release granules, delayed release granules, or both extended and delayed release granules of the powder blend may be milled to achieve a desired size range. The particle size range of the extended release granules, delayed release granules, or both extended and delayed release granules of the powder blend may also impact the buoyancy of the granules. For example, surface tension plays a role in the buoyancy of the extended release granules, delayed release granules, or both extended and delayed release granules of the powder blend once dispersed in a dispersion medium. If the particle size of the granules is too small, the excess surface tension makes it difficult for the granules to suspend and they will only float on top of the dispersion medium. Likewise, if the surface tension is too large, the reduced surface area results in granules that sink in the dispersion medium. In one aspect, to achieve the desired neutral buoyancy of the extended release granules in a dispersion medium (e.g. aqueous medium), delayed release granules, or both extended and delayed release granules, the granules can be milled or passed through a sieve to remove agglomerates after granulation and to provide a particle size ranging from 150 μm to 1200 μm, or any value or range therebetween. In other aspects, the granules can be milled or passed through a sieve to remove agglomerates after granulation and to provide a particle size ranging from 425 μm to 850 μm, or any value or range therebetween. These particles can be either regularly or irregularly shaped. These particle sizes may be determined using sieve analysis through a sieve shaker having USP standard wire mesh sieves conforming to ASTM specifications (e.g. 16, 20, 30, 40, 60, or 80 mesh screen, optionally a scree of 10 to 80 mesh). A particle prior to coating is optionally sized to 10 to 30 mesh or any value or range therebetween, optionally 30 mesh, 25 mesh, 20 mesh, 18 mesh, 16 mesh, 14 mesh, 12 mesh, or 10 mesh.

In certain aspects, the extended release granules, delayed release granules, or both extended and delayed release granules optionally includes one or more additives including but not limited to, e.g., one or more of a diluent, binder, lubricant, disintegrant, stabilizer, surfactant, glidant, sweetener, a preservative, sodium citrate; silica; flavoring agents, coloring agents, preservatives, or other components. The choice of which such materials to us, if any, and the amounts to be utilized are considered to be within the abilities of one of skilled in the art, in view of the disclosure herein. However, additives which might adversely affect the neutral buoyancy of the extended release granules, delayed release granules, or both extended and delayed release granules, should either not be used or only be used in quantities insufficient to cause a substantial negative effect upon the neutral buoyancy or other characteristics of the composition.

Exemplary diluents may include, but are not limited to calcium carbonate, calcium phosphate dibasic, calcium phosphate tribasic, dimagnesium phosphate, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol

Exemplary binders may include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Examples of polyvinylpyrrolidone include povidone, copovidone and crospovidone.

Exemplary lubricants may include, but are not limited to magnesium stearate, calcium stearate, stearic acid, and hydrogenated vegetable oil (e.g. comprising hydrogenated and refined triglycerides of stearic and palmitic acids).

Exemplary disintegrants may include, but are not limited to starches, sodium starch glycolate, croscarmellose sodium, clays, celluloses, algins, gums, or crosslinked polymers (e.g., crosslinked polyvinyl pyrrolidone), alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, sodium bicarbonate, pregelatinized starch, low-substituted hydroxypropyl cellulose.

Fillers include, for example, materials such as kaolin, powdered cellulose, and microcrystalline cellulose, as well as soluble materials such as mannitol, urea, sucrose, lactose, lactose monohydrate, dextrose, sodium chloride, sodium bicarbonate, and sorbitol.

Exemplary sweeteners may include those sweeteners well known in the art, including both natural and artificial sweeteners. Thus, exemplary sweeteners may include water-soluble sweetening agents such as monosaccharides, disaccharides, and polysaccharides such as xylose, ribose, glucose, mannose, galactose, fructose, high fructose corn syrup, dextrose, sucrose, sugar, maltose, partially hydrolyzed starch, or corn syrup solids and sugar alcohols such as sorbitol, xylitol, mannitol and mixtures thereof. Additional exemplary sweeteners include optionally sugar or sugar substitute (e.g. sucralose (1,6-Dichloro-1,6-dideoxy-β-D-fructofuranosyl-4-chloro-4-deoxy-α-D-galactopyranoside), aspartame, acesulfame potassium, and the like.

Exemplary preservatives may include sodium benzoate, sodium bicarbonate, benzoic acid, potassium sorbate, sea salt (optionally in the form of ancient sea salt available as Redmond's Real Sea Salt), salts of edetate (also known as salts of ethylenediaminetetraacetic acid, or EDTA, such as disodium EDTA), carnosic acid, parabens (e.g., methyl, ethyl, propyl or butyl-hydroxybenzoates, etc.), and sorbic acid. Other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3-PDTA); 2,2-ethylenedioxybis[ethyliminodi(acetic acid)] (EGTA); 1,10-bis(2-pyridylmethyl)-1,4,7,10-tetraazadecane (BPTETA); ethylenediamine (EDAMINE); Trans-1,2-diaminocyclohexane-N,N,N′,N′-tetraacetic acid (CDTA); ethylenediamine-N,N′-diacetate (EDDA); phenazine methosulphate (PMS); 2,6-Dichloro-indophenol (DCPIP); Bis(carboxymethyl)diaza-18-crown-6 (CROWN); porphine; chlorophyll; dimercaprol (2,3-Dimercapto-1-propanol); citric acid; tartaric acid; fumaric acid; malic acid (optionally in the form of DL-malic acid); and salts thereof can be utilized as preservatives. Each preservative must be evaluated in each formulation to assure the compatibility and efficacy of the preservative. Methods for evaluating the efficacy of preservatives in compositions and formulations are known to those skilled in the art.

Exemplary flavorings may include both natural and artificial flavors, and mints such as peppermint, menthol, artificial vanilla, vanilla extract, ginger extract (optionally in the form of ginger CO2 Extract), chocolate, cinnamon, citrus (optionally including D-limonene oil), various fruit flavors, both individual and mixed, tea flavors, sweetness modifiers, essential oils (i.e. thymol, eucalyptol, menthol, theacrine, and methyl salicylate), pie flavors (e.g., pumpkin pie, lemon pie, apple pie), herbs (e.g., rosemary, lemongrass, lavender, jasmine, lemon verbena; optionally including carnosic acid), and extracts thereof, and the like. Exemplary fruit flavorings may include both natural and artificial flavors, such as blueberry, raspberry, strawberry, grape, lemon, lime, cherry, mango, grapefruit, coconut, apple, pineapple, and the like. Flavoring agents may be utilized to provide an appealing flavor to the compositions, mask other flavors of the compositions (e.g., a vegan protein masker), enhance the sweetness and/or savoriness of the compositions, and the like.

Exemplary coloring agents may include both natural and artificial colorants such as spirulina extract, turmeric powder, fruit powder, vegetable powder, cocoa powder, paprika extract, beetroot, and the like. Exemplary colors that may be provided by one or more coloring agents include red, blue, yellow, green, orange, purple, pink, gray, black, white, brown, variations thereof, and the like.

In one aspect, the extended release granules, delayed release granules, or both extended and delayed release granules are formed into a solid unit dose or solid preparation. Such granules may take the form of a powder.

In some aspects, a performance-enhancing composition is in the form of a powder, optionally an immediate release powder. Optionally, a performance-enhancing composition includes one or more beads, beadlets, capsules, tablets (e.g. oral disintegrating tablet), or other forms suitable for oral administration to a subject.

In some aspects, the above-described immediate release powder and extended release granules, delayed release granules, or both extended and delayed release granules, are optionally suspended, diluted, solubilized, or otherwise combined with a dispersion medium to provide an oral suspension. Thus, in certain aspects, a performance-enhancing immediate and extended release oral suspension comprising an immediate release component comprising at least one performance-enhancing component; an extended release granule component comprising a core, the core comprising at least one performance-enhancing component and one or more fatty materials; and a dispersion medium is provided. A dispersion medium is optionally an aqueous solution or other liquid. Any suitable aqueous solution may be used, such as water, milk, fruit, juice, alcohol, or the like. Organic solutions may or may not also be used for the dispersion medium. The dispersion medium is optionally non-toxic or used in a non-toxic amount. In some aspects, the dispersion medium may include water a from 75 wt. % to 99.5 wt. % of the dispersion medium, or any value or range in between. In some aspects, a dispersion medium consists of water. In some aspects, a dispersion medium optionally excludes a pH modifier. A dispersion medium optionally excludes a thickening agent defined herein as a material that increases the viscosity of the dispersion medium relative to a dispersion medium absent the thickening agent whereby a thickening agent is not a performance-enhancing supplement. A dispersion medium following combination with a first and second component optionally has a pH of 2.0 to 9.0, optionally a pH in excess of 5.5, optionally a pH of from 6.5 to 8.0.

The instantly-disclosed performance-enhancing compositions can be administered by any desirable route. Optionally, the composition is administered orally. An administration time is optionally before, during or following exercise. Optionally, the composition is administered orally prior to exercise or during exercise.

In some aspects, a composition is administered by adding a dispersion medium, optionally water, to a substantially dry mixture of first immediate release component and optional second delayed or extended release component. A first component optionally fully or partially solubilizes in the dispersion medium and the second component is suspended in the dispersion medium for such a time sufficient for a subject to orally consume the composition. As such processes are provided for enhancing athletic performance or preventing fatigue that include administering a the instantly-disclosed composition(s) as provided to a mammalian subject, optionally a human, wherein the administration is performed at a time suitable for enhancing athletic performance or preventing fatigue. Thus, processes are provided for maintaining vasodilation during and after a workout, stimulating muscle synthesis and repair over an extending period of time, and/or preventing athletic fatigue, such as sustaining energy levels and avoiding a subsequent energy level crash, that include administering the instantly-disclosed immediate and extended release performance-enhancing supplement compositions. An administration time is optionally from 0 to 30 minutes prior to exercise or other athletic activity, during athletic activity, or combinations thereof.

In some aspects, a method of improving mitochondrial activity includes administering a performance-enhancing composition comprising an immediate release powder comprising at least one first performance-enhancing component and optionally an acid. The immediate release powder is partially or soluble in an aqueous medium at 25 degrees Celsius. This method may include any of the otherwise described embodiments of this disclosure.

In aspects, a method of improving mitochondrial activity includes administering a performance-enhancing composition comprising a formulation including an outer capsule housing an edible oil, medium chain triglycerides, or a performance-enhancing component wherein one or more inner capsules or beads are included in the outer capsule and the inner capsule or beads include one or more performance-enhancing components. The formulation optionally includes an inner capsule or bead and an outer capsule, wherein the outer capsule and the inner capsule or bead are separated by a barrier. The barrier between the outer capsule and the inner capsule or bead of the formulation is optionally formed by the exterior of the inner capsule or bead. This method may include any of the previously described embodiments of this disclosure.

The compositions as provided herein may be administered in a dose sufficient to increase mitochondrial ATP levels, increase the presence of or membrane transport of molecular hydrogen (e.g. hydrogen gas) into a cell or organelle, or combinations thereof. Optionally, a composition is dosed to a subject at a dose of 1 mg to 30 grams or more. Optionally, the performance-enhancing composition is administered to a subject in an amount that is at least 100 mg, 200 mg, 500 mg, 800 mg, or more. Optionally, the performance-enhancing composition is administered to a subject in an amount that is at least 1 gram, 2 grams, 5 grams, 10 grams, 15 grams, or more.

A performance-enhancing composition optionally is administered prior to exercise. Optionally, a composition is administered to a subject prior to exercise by 1 minute or more, optionally 5 minutes or more, optionally 10 minutes or more prior to exercise. Optionally, a performance-enhancing composition is administered to a subject during exercise. Optionally, a performance-enhancing composition is administered to a subject before exercise and during exercise.

The foregoing description is illustrative of particular aspects of the invention, but is not meant to be a limitation upon the practice thereof. In order that various aspects may be more readily understood, reference is made to the following examples which are intended to illustrate various aspects, but do not limit the scope thereof.

### EXAMPLES

### Example 1

### Method of Forming the Immediate Release Powder

The immediate release powder of the instant compositions optionally includes the components as found in Table A, including the at least one performance-enhancing component.

TABLE A
Performance-Enhancing Components
in the Immediate Release Powder

| Ingredient | Weight % of Granule |
|---|---|
| Macromineral | 0%-20% |
| Trace Mineral | 0%-60% |
| Plant Component | 0%-25% |
| Citrulline | 0%-20% |
| Amino Acid | 0%-20% |
| Citric Acid | 0%-60% |
| Malic Acid | 0%-25% |
| Tartaric Acid | 0%-10% |

### Example 2

### Method of Granulating and Forming the Extended Release Granules

The extended release granules and/or delayed release granules, if further included, are made using a high shear granulation process, followed by a coating process. The desired time release, e.g. sustained release or delayed release is achieved by both the granulation composition and/or the coating.

The high shear process typically begins by adding the dry powders of the formulation to the high shear granulator, which is a sealed “mixing bowl” with an impellor that rotates through the powder bed, and a chopper blade that breaks up over-agglomerates that can form during the process. There are typically three phases to the high shear process, dry mixing, solution addition, or wet massing and high shear granulation.

In the first phase, dry powders, such as the at least one performance-enhancing component (Table A), the one or more fatty materials (Table B), optionally the one or more swellable polymers (Table C), and various optional additives are mixed together by the impeller blade which rotates through the powder bed, creating a “roping” vortex of powder movement. The one or more fatty acids, which can be low density fatty powders, are optionally granulated the at least one performance-enhancing component with low density fatty powders—to target a bulk density of the final granule formulation will be from 0.30 g/cc to 0.75 g/cc, including any value or range therebetween, so that the final granule formulation will be suspendable and will exhibit neutral buoyancy in an aqueous medium. The one or more swellable polymers are also optionally granulated with the at least one performance-enhancing component to modify, prolong, and/or slow the release over time of the at least one performance-enhancing component from the granules. The dry mixing phase typically lasts for only a few minutes.

In the second phase of the process, a granulating liquid is added to the sealed product container, usually by use of a peristaltic pump. The solution can contain a binder with sufficient viscosity to cause the wet massed particles to stick together or agglomerate. However, a binder may be incorporated only in the granulating solution or only in the dry powder. If the binder is only in the dry powder, then water is used as the granulating solution. It is common for the solution addition phase to last over a period of from three to five minutes. While the impeller is rotating rather slowly during this step of the process, the chopper blade is turning at a fairly high rate of speed to chop up over-sized agglomerates, while not interfering with the impellers movement.

Once the binder solution is added to the product container, the final stage of the granulation process begins. In this phase, high shear forces are generated as the impeller blades push through the wet massed powder bed, further distributing the binder and intimately mixing the ingredients contained therein until the desired granule particle size and density end-points are reached. Granule particle size and density end-points are often determined by the power consumption and/or torque on the impeller. The extended release granules and delayed release granules will have a particle size ranging from 150 μm to 1200 μm, or any value or range therebetween. Similarly, the granules can be milled or passed through a sieve to remove agglomerates after granulation and to provide a particle that will have a particle size ranging from 425 μm to 850 μm, or any value or range therebetween.

Once the high shear granulation process is completed, the material is transferred to a fluid bed dryer, or alternatively, can be spread out onto trays that are then placed in a drying oven, where the product is dried until the desired moisture content is achieved.

Once dried, the granules undergo a coating process. The barrier coating (Table D) confers either extended release (e.g., ethylcelullose based coating) or delayed release properties (e.g. an enteric coating such as a shellac coating) to the granules. For coating, the barrier coat can be applied, e.g., as either an aqueous suspension or dispersion, over the extended release granules, delayed release granules, or both extended and delayed release granules, and forms a separate layer thereon.

The extended and delayed release granules of the instant compositions optionally includes the components as found in Tables A-E, including the at least one performance-enhancing component (Table B), the one or more fatty materials (Table C), optionally the one or more swellable polymers (Table D), and optionally the barrier coating (Table E).

TABLE B
Performance-Enhancing Components in the
Extended and/or Delayed Release Granules

| Ingredient | Weight % of Granule |
|---|---|
| Betaine Anhydrous (Trimethyl Glycine) | 0%-30% |
| Betaine | 0%-20% |
| Caffeine | 0%-25% |
| Carnitine | 0%-20% |
| Acetyl-L-Carnitine | 0%-20% |
| N-Acetyl L-Tyrosine | 0%-15% |
| Niacin/Nicotinic Acid Vit B3 | 0%-10% |
| Vitamin B12 | 0%-5% |
| Vitamin D3 | 0%-5% |
| Citrus Auranium | 0%-5% |
| Citrulline | 0%-40% |
| Citrulline Malate | 0%-40% |
| L-Citrulline DL-Malate 2:1 | 0%-30% |
| Creatine | 0%-20% |
| Alpha Yohimbine | 0%-2% |
| Leucine | 0%-60% |
| Isoleucine | 0%-25% |
| Theanine | 0%-10% |
| Valine | 0%-25% |
| Glutamine | 0%-25% |
| Agmatine Sulfate | 0%-15% |
| Arginine Silicate Inositol | 0-15% |
| Pepform BCAA 2:1:1 | 0%-15% |
| Astragalus membranaceus and Panax | 0%-5% |
| notoginseng | |
| Beta-Alanine | 0%-40% |
| Norvaline | 0%-50% |
| Niacin Usp/Vitamin B3 | 0%-15% |
| Grape Seed Extract | 0%-15% |
| Piper Nigrum Fruit Extract | 0%-2% |
| Ashwagandha Extract | 0%-25% |

TABLE C
Granule Fatty Material

| Ingredient | Weight % of Granule |
|---|---|
| Hydrogenated Vegetable Oil | 15%-60% |
| Stearic acid | 15%-60% |
| Fractionated Vegetable Oils | 15%-60% |
| Fatty Acid Esters of Glycerol | 15%-60% |

TABLE D
Granule Swellable Polymers

| Ingredient | Weight % of Granule |
|---|---|
| Hydroxypropyl methylcellulose | 0%-25% |
| Microcrystalline cellulose | 0%-25% |
| Ethylcellulose | 0%-25% |
| Methylcellulose | 0%-25% |
| Gums | 0%-25% |

TABLE E
Granule Coating

| Ingredient | Weight % of Granule |
|---|---|
| Ethylcellulose | 0%-10% |
| Shellac | 0%-10% |
| Hydroxypropyl methylcellulose | 0%-10% |

The extended and delayed release granules of the instant compositions are subsequently mixed with the immediate release powder that includes at least one performance-enhancing component (Table B). The performance-enhancing component of the extended release granules, delayed release granules, or both extended and delayed release granules includes the same, different, additional, or fewer at least one performance-enhancing component of relative to the at least one performance-enhancing component of the immediate release powder. The extended release granules and/or delayed release granule and the immediate release powder can be mixed to form a powder blend, or can be combined in a dispersion medium (e.g. an aqueous medium such as water or sports drink) to form an oral suspension.

### Example 3

### Forming Extended Release Granules

Extended release granules are formed by high shear mixing substantially as understood in the art. Briefly, one or more performance-enhancing components (e.g. caffeine, beta alanine (e.g. CAROSYN), trimethyl glycine, creatine, tyrosine, niacin or derivatives, plant extract (e.g. grape seed extract or Piper nigrum), norvaline, leucine, BCAA, methylcobalamin, tyrosine, theanine, and/or others as described herein) is charged to a Littleford W-10 shearmixer with a hot water jacket to allow circulating hot water to keep the vessel hot. After mixing for 1 minute at 1000 RPM, spray chilled fat powder (Sterotex HM®) is added to the vessel. The work input is increased to 2000 RPM and then adjusted down to 600 RPM for 5 minutes. The resulting particles are sized to 10-30 mesh. Extended release granules can include an extended release composition (e.g. HPMC, ethylcellulose, microcrystalline cellulose), and can further include an optional extended release material coated onto the surface. Likewise, extended release granules in which an extended release composition (e.g. HPMC, ethylcellulose, microcrystalline cellulose) is not added to the granule itself, the extended release material may be coated onto the surface.

The granules are subjected to dissolution testing for release of the active into a dispersion medium using the basket method. Briefly, the granules are placed in water housed in a basket and rotated (25-150 RPM) at a constant temperature (35° C.). Samples are drawn at various time intervals and subjected to analysis. Granules employing caffeine are formed to have a release profile of NMT 20% 20 min and NLT 80% 60 min. In other aspects, granules employing caffeine are formed to have a release profile of NMT 10% 20 min and NLT 80% 40 min. Granules employing BCAA are formed to have a release profile of NMT 20% 1 hour and NLT 80% 4 hours. In other aspects, granules employing BCAA are formed to have a release profile of NMT 10% 1 hour, NMT 30% in 2 hours, and NLT 80% 4 hours. Granules employing norvaline have a release profile of NMT 70% at 30 min.

### Example 4

### Immediate Release Performance-Enhancing Composition

The formulation of the instant compositions optionally includes the components as found in Table F in powder form, including the at least one performance-enhancing component and a trace mineral.

TABLE F
Performance-Enhancing Components in the Formulation

| Ingredient | Weight % of Formulation |
|---|---|
| Macromineral | 0%-20% |
| Trace Mineral | 0%-60% |
| Plant Component | 0%-20% |
| Amino Acid | 0%-20% |
| Cittruline | 0%-20% |
| Citric Acid | 0%-60% |
| Malic Acid | 0%-25% |
| Tartaric Acid | 0%-10% |
| Vitamin | 0%-15% |
| Testosterone Booster | 0%-30% |
| Stimulant | 0%-25% |

### Example 5

### Multicomponent Formulation

The formulation, if further included, is optionally made by loading the contents of the inner capsule into an inner gelatin capsule and sealing the inner gelatin capsule. Then, the inner gelatin capsule is loaded into a larger, outer capsule. Next, the components of the outer capsule (optionally including beadlets) are loaded into the outer capsule, and the outer capsule is sealed.

The formulation is optionally made by loading the contents of the inner capsule into an inner gelatin capsule and sealing the inner gelatin capsule. Next, the components of the outer capsule (optionally including beadlets) are loaded into the outer capsule. Then, the inner gelatin capsule is loaded into a larger, outer capsule. Subsequently, the outer capsule is sealed.

The inner and outer capsules of the instant compositions optionally includes the components as found in Table F, including the at least one performance-enhancing component (Table F). The outer capsules optionally contain one or more of the components in Table G. The inner capsules optionally contain one or more of the components in Table H.

TABLE G
Components Optionally Included in the Outer Capsule

| Ingredient | Weight % of Formulation |
|---|---|
| Caffeine | 0%-50% |
| Edible Oil | 0%-50% |
| Ginger Extract | 0%-5% |
| Limonene oil | 0%-1% |
| Rosemary Extract | 0%-1% |
| Vitamin | 0%-5% |
| Medium chain triglycerides | 0%-90% |
| Pink Ashwagandha | 0%-50% |
| Edible Oil | 0%-50% |

TABLE H
Components Optionally included in the Inner Capsule

| Ingredient | Weight % of Formulation |
|---|---|
| Magnesium-based Compound | 0%-50% |
| Betaine Anhydrous (Trimethyl | 0%-30% |
| Glycine) | |
| Betaine | 0%-20% |
| Boron citrate | 0%-3% |
| Trace mineral | 0%-60% |
| Carnitine | 0%-20% |
| Acetyl-L-Carnitine | 0%-20% |
| N-Acetyl L-Tyrosine | 0%-15% |
| Niacin/Nicotinic Acid Vit B3 | 0%-10% |
| Vitamin B12 | 0%-5% |
| Citrus Auranium | 0%-5% |
| Citrulline | 0%-40% |
| Citrulline Malate | 0%-40% |
| L-Citrulline DL-Malate 2:1 | 0%-30% |
| Creatine | 0%-20% |
| Alpha Yohimbine | 0%-2% |
| Leucine | 0%-60% |
| Isoleucine | 0%-25% |
| Theanine | 0%-10% |
| Valine | 0%-25% |
| Glutamine | 0%-25% |
| Agmatine Sulfate | 0%- 15% |
| Arginine Silicate Inositol | 0-15% |
| Pepform BCAA 2:1:1 | 0%-15% |
| Astragalus membranaceus and Panax | 0%-5% |
| notoginseng | |
| L-alpha glycerylphoroylcholine | 0% to 5% |
| Beta-Alanine | 0%-40% |
| Niacin Usp/Vitamin B3 | 0%-15% |
| Piper Nigrum Fruit Extract | 0%-2% |
| Ashwagandha Extract | 0%-25% |
| Diindolymethane | 0%-3% |
| Forskholin | 0%-2% |
| Shilajit | 0%-10% |
| Eurycoma longfolia | 0%-10% |
| Chromium | 0%-10% |
| Agaricus bisporus | 0%-10% |
| Theacrine | 0%-10% |
| Capsaicinoid | 0%-10% |
| Huperzine A | 0%-1% |
| Lions Mane Extract | 0%-10% |
| Berberine | 0%-10% |
| Coleus Forskohil | 0%-10% |
| Microcrystalline Cellulose | 0%-10% |
| Silicon Dioxide | 0%-10% |
| L-α-glycerophosphocoline | 0%-10% |
| Shilajit | 0%-70% |
| Urycoma Longifolia | 0%-50% |
| Mucuna Pruriens | 0%-30% |

The components listed in tables A-H are understood to be non-exhaustive and merely exemplary or illustrative examples of components that optionally comprise the performance-enhancing composition.

### Example 6

### Measuring Exercise Performance

Ten subjects were selected to evaluate athletic performance changes by administration of a performance-enhancing composition as provided in Example 4. The subjects included both make and females and subjects that regularly train in a gym or typically are sedentary. All subjects were asked to run for 52 minutes on a relatively flat surface. Parameters such as caloric output and heart rate were measured using a gps and heart rate monitor to measure total calories burned (kcal) and average heartrate during the exercise period. After at least 24 hours rest, the subjects consumed 12.6 grams of the composition of Example 4 diluted in 812 ounces of water. Consumption was 10 prior to a repeat of the same course for the same 52 minute duration. The results of the evaluation are presented in Table I.

TABLE I
Exercise performance output

| | |
|---|---|
| CALORIES (kcal) | Heartrate (bpm) |

| Subject | No Supp | Supp | No Supp | Supp |
|---|---|---|---|---|
| 1 | 719 | 894 | 148 | 168 |
| 2 | 857 | 874 | 163 | 162 |
| 3 | 805 | 831 | 158 | — |
| 4 | 741 | 775 | 151 | 154 |
| 5 | 731 | 815 | 151 | 156 |
| 6 | 705 | 760 | 152 | 159 |
| 7 | 627 | 564 | 154 | 148 |
| 8 | 784 | 825 | 154 | 156 |
| 9 | 805 | 957 | 158 | 175 |
| 10 | 680 | 748 | 146 | 150 |

The subjects all showed increases in calories burned and average heartrate during the exercise period indicating increases in exercise output. Statistical analyses using the student's t-test revealed an average increase in calories burned of 58.9 kcal (p=0.01) and average heartrate increase of 5.1 (p=0.4). The results demonstrate that consuming the composition of Example produced a statistically significant increase in exercise performance.

Various modifications of the present invention, in addition to those shown and described herein, will be apparent to those skilled in the art of the above description. Such modifications are also intended to fall within the scope of the appended claims.

It is appreciated that all reagents are obtainable from commercial sources known in the art unless otherwise specified.

This description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the compositions or processes are described as using specific materials or an order of individual steps, it is appreciated that materials or steps may be interchangeable such that the description of the invention may include multiple parts or steps arranged in many ways as is readily appreciated by one of skill in the art.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular aspects of the invention, but is not meant to be a limitation upon the practice thereof.

1. A method of increasing mitochondrial activity in a subject comprising:
administering to a subject in need a performance-enhancing composition comprising an acid and at least one first performance-enhancing component comprising a macromineral;
wherein the performance-enhancing composition is fully soluble in an aqueous medium or is partially soluble in the aqueous medium such that the performance-enhancing composition is maintained in the aqueous medium for a sufficient period of time for the subject to consume the performance-enhancing composition in the aqueous medium.

2. The method of claim 1, wherein the macromineral is boron, calcium, chromium, cobalt, copper, fluorine, iodine, iron, magnesium, manganese, molybdenum, nickel, selenium, silicon, sodium, vanadium, zinc, or combinations thereof.

3. The method of claim 1, wherein the macromineral is calcium or magnesium.

4. The method of claim 1, wherein the at least one first performance-enhancing component comprises a magnesium-based composition.

5. The method of claim 4, wherein the magnesium-based composition comprises at least one fatty acid ester.

6. The method of claim 4, wherein the magnesium-based composition comprises magnesium carbonate, magnesium citrate, a liposomal magnesium oxide complex, or combinations thereof.

7. The method of claim 1, wherein the performance-enhancing composition comprises a magnesium-based composition, and one or more of a trace mineral complex, citric acid, malic acid, tartaric acid, or combinations thereof.

8. The method of claim 1, wherein the performance-enhancing composition further comprises extended release granules comprising a core, the core comprising at least one second performance-enhancing component and one or more fatty materials.

9. The method of claim 8, wherein the extended release granules further comprise a barrier coating layer.

10. The method of claim 1, wherein the performance-enhancing composition further comprises an alkaline agent suitable to increase the alkalinity within a subject or portion thereof.

11. A performance-enhancing composition comprising:
an outer capsule, the outer capsule comprising an edible oil, a triglyceride in the form of an oil, at least one first performance-enhancing component or a combination thereof;
an inner capsule or bead, the inner capsule or bead comprising a second performance-enhancing component, the inner capsule or bead housed within the outer capsule.

12. The performance-enhancing composition of claim 11, wherein the at least one first performance-enhancing component, the second performance-enhancing component or both comprises a magnesium-based composition.

13. The performance-enhancing composition of claim 12, wherein the magnesium-based composition is selected from the group consisting of magnesium oxide, magnesium citrate, a magnesium bisglycinate, elemental magnesium, or combinations thereof.

14. The performance-enhancing composition of claim 12, wherein the magnesium-based composition comprises at least one fatty acid ester.

15. The performance-enhancing composition of claim 12, wherein the at least one second performance-enhancing component comprises the magnesium-based composition, a trace mineral complex, theacrine, a plant component, a stimulant, testosterone booster, or combinations thereof.

16. The performance-enhancing composition of claim 12, wherein the at least one first performance-enhancing component comprises a testosterone booster or first stimulant, and the second performance-enhancing component comprises the magnesium-based composition, the inner capsule or bead further comprising a second stimulant.

17. The performance-enhancing composition of claim 11, wherein the second performance-enhancing component is selected from the group consisting of boron, calcium, chromium, cobalt, copper, fluorine, iodine, iron, magnesium, manganese, molybdenum, nickel, selenium, silicon, sodium, vanadium, zinc, or combinations thereof.

18. The performance-enhancing composition of claim 11, wherein the outer capsule comprises the edible oil and the triglyceride, and the second performance-enhancing component comprises a magnesium-based composition.

19. A method of increasing exercise performance of a subject comprising administering to the subject the performance-enhancing composition of claim 11.

20. The method of claim 19 wherein the outer capsule comprises the edible oil and the triglyceride, and the second performance-enhancing component comprises a magnesium-based composition, and a stimulant.
